(19) United States
(12) Patent Application Publication
(10) Pub. No.: US 2009/0264498 A1
(43) Pub. Date: Oct. 22, 2009

(54) Cathepsin C Inhibitors

(76) Inventors: Dramane Ibrahim Laine, King of Prussia, PA (US); Guoliang Lin, King of Prussia, PA (US); Brent W. McCleland, King of Prussia, PA (US); Michael R. Palovich, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/424,761

(22) Filed: Apr. 16, 2009

Related U.S. Application Data
(60) Provisional application No. 61/046,047, filed on Apr. 18, 2008.

(51) Int. Cl. A61K 31/40; C07D 207/14

(52) U.S. Cl. 514/426; 548/557

(57) ABSTRACT

This invention relates to compounds of formula (I)

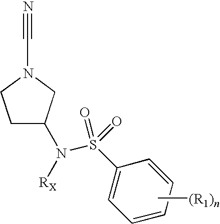

which are useful for treating chronic obstructive pulmonary disease, and the like.

### CROSS REFERENCE TO PRIOR APPLICATIONS

This application claims the benefit to U.S. provisional 61/046,047 filed 18 Apr. 2008.

### FIELD OF THE INVENTION

This invention relates to certain 1-cyano-3-pyrrolidinyl-N-substituted sulfonamides that are cathepsin C inhibitors, and their use in the treatment of diseases mediated by the cathepsin C enzyme such as chronic obstructive pulmonary disease.

### BACKGROUND OF THE INVENTION

Cathepsins are a family of enzymes included in the papain superfamily of cysteine proteases. Cathepsins B, C, F, H, K, L, S, V, and X have been described in the scientific literature. Cathepsin C is also known in the literature as Dipeptidyl Peptidase I or “DPPI.”

A number of recently published studies have begun to describe the role cathepsin C plays in certain inflammatory processes. See E.g. Adkison et al., The Journal of Clinical Investigation 109:363-371 (2002); Tran et al., Archives of Biochemistry and Biophysics 403:160-170 (2002); Thiele et al., The Journal of Immunology 158: 5200-5210 (1997); Bidere et al., The Journal of Biological Chemistry 277: 32339-32347 (2002); Mabee et al., The Journal of Immunology 160: 5880-5885; McGuire et al., The Journal of Biological Chemistry, 268: 2458-2467; and Paris et al., FEBS Letters 369: 326-330 (1995). From these studies, it appears that cathepsin C is co-expressed with certain serine proteases, which are released from inflammatory cells recruited to cites of inflammation, and acts as a physiological activator of these proteases. Once activated, these proteases are capable of degrading various extracellular matrix components, which can lead to tissue damage and chronic inflammation.

For example, Chronic Obstructive Pulmonary Disease (“COPD”) is a chronic inflammatory disease where cathepsin C appears to play a role. The American Thoracic Society defines COPD as “a disease characterized by the presence of airflow obstruction due to chronic bronchitis or emphysema; the airflow obstruction is generally progressive, may be accompanied by airway hyperreactivity, and may be partially reversible.” American Journal of Respiratory and Critical Care Medicine 152: S77-S120 (1995). Chronic bronchitis is generally characterized by a chronic productive cough, whereas emphysema is generally characterized by permanent enlargement of the airspaces distal to the terminal bronchioles and airway wall destruction. Chronic bronchitis and emphysema usually occur together in COPD patients.

Cigarette smoking is a significant risk factor for developing COPD. Exposure to cigarette smoke and other noxious particles and gases may result in chronic inflammation of the lung. In response to such exposure, inflammatory cells such as CD8+ Tcells, macrophages, and neutrophils are recruited to the area. These recruited inflammatory cells release proteases, which are believed to play a major role in the disease etiology by degrading airway walls. Proteases believed to be involved in this process include the serine proteases neutrophil elastase (“NE”), chymase (“CY”), cathepsin G, proteinase 3 and granzymes A and B. Cathepsin C appears to be involved in activating these enzymes.

Rheumatoid arthritis (“RA”) is another chronic inflammatory disease where cathepsin C appears to play a role. Neutrophils are recruited to the site of joint inflammation and release cathepsin G, NE, and proteinase 3, which are believed to be responsible for cartilage destruction associated with RA. Cathepsin C appears to be involved in activating these enzymes.

Other conditions where cathepsin C may play a role include osteoarthritis, asthma, and Multiple Sclerosis. See E.g. Matsui K. Yuyama N. Akaiwa M. Yoshida N L. Maeda M. Sugita Y. Izuhara K., Identification of an alternative splicing variant of cathepsin C/dipeptidyl-peptidase I, Gene. 293(1-2): 1-7, 2002 Jun. 26; Wolters P J. Laig-Webster M. Caughey G H., Dipeptidyl peptidase I cleaves matrix-associated proteins and is expressed mainly by mast cells in normal dog airways, American Journal of Respiratory Cell & Molecular Biology. 22(2): 183-90, 2000.

One approach to treating these conditions is to inhibit the activity of the serine proteases involved in the inflammatory process, especially NE activity. See E.g., Ohbayashi, “Neutrophil elastase inhibitors as treatment for COPD”, Expert Opin. Investig. Drugs 11(7): 965-980 (2002); Shapiro, “Neutrophil Elastase: Path Clearer, Pathogen Killer, or Just Pathologic?”, Am. J. Respir. Cell Mol. Biol. 26: 266-268 (2002). In light of the role cathepsin C plays in activating certain serine proteases, especially NE, it is desirable to prepare compounds that inhibit its activity, which thereby inhibit serine protease activity. Thus, there is a need to identify compounds that inhibit cathepsin C, which can be used in the treatment of a variety of conditions mediated by cathepsin C.

### SUMMARY OF THE INVENTION

In a first instance this invention relates to compounds of formula (I)

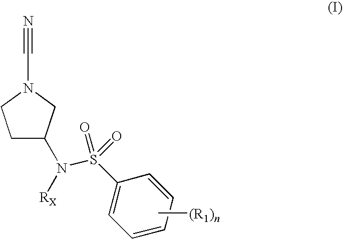

or a salt thereof wherein

Rₓ is C₁-C₆alkyl; or aryl or arylC₁-C₆alkyl where aryl is unsubstituted or substituted by halo, C₁-C₆alkyl, halo-substituted-C₁-C₆alkyl, C₁-C₆alkoxy optionally substituted with halo, or S(O)_{y} where y is 0-2;

n is 1-5,

each R₁ is independently halo; OR₂; C₁-C₁₀alkyl unsubstituted or substituted by halo; CN; C(O)NR₃R₄; NO₂; NHC(O)NR₅R₆; NR₇R₈; a heteraromatic ring containing 1-3 heteroatoms selected from the group consisting of N, O or S; aryl, unsubstituted or substituted by halo or C₁-C₆ alkyl; C₃-C₆cycloalkyl; C₄-C₆cycloalkenyl; C₁-C₁₀alkenyl; C₁-C₁₀alkynyl; or C(O)OR₂;

R₂ is H, C₁-C₆ alkyl, haloC₁-C₆ alkyl, C(O)R₉;

R₃ is H; C₁-C₆ alkyl; C₃-C₆cycloalkyl; or C₃-C₆heterocycloalkyl containing N, O or S and where when the ring heteroatom is N it is optionally substituted by CN;

R₄ is H, or C₁-C₆ alkyl;

R₅ and R₆ are independently H; C₁-C₆alkyl; aryl unsubstituted or substituted by halo, C₁-C₄alkoxy, C₁-C₄alkyl, or OR₂; C₁-C₁₀ alkyl unsubstituted or substituted by halo or C₁-C₄alkoxy; C₃-C₆cycloalkyl; C₃-C₆cycloalkylalkyl; C₁-C₁₀alkenyl; C₁-C₁₀alkynyl; C₃-C₆heteroycloalkyl; C₃-C₆heteroycloalkyl C₁-C₆alkyl; heteroarylC₁-C₆alkyl or heteroaryl wherein the heteroaryl ring is unsubstituted or substituted by halo, C₁-C₆ alkyl or halo-substituted-C₁-C₆ alkyl; or arylC₁-C₆alkyl wherein the aryl group is unsubstituted or substituted by halo, OR₂, or C(O);

R₇ and R₈ are independently H; C₁-C₁₀alkyl; arylC₁-C₆alkyl wherein the aryl group is unsubstituted or substituted by R₉, halo, or C₁-C₆alkyl; C₁-C₁₀alkyl substituted by C₃-C₆cycloalkyl, one or more OH groups, halo; heteroarylC₁-C₆alkyl; or heteroaryl; or

R₉ is OH, C₁-C₆ alkoxy or NR_{2′}R_{3′}, wherein R_{2′} or R₃ are independently H or C₁-C₆alkyl.

In a second instance, this invention relates to the use of a compound of formula (I) or a pharmaceutically acceptable salt thereof in the prevention, management or treatment of a respiratory or inflammatory disease, such as chronic obstructive pulmonary disease or rhinitis.

In a further aspect, this invention relates to a pharmaceutically acceptable formulation comprising a compound of formula (I) or a pharmaceutically acceptable salt thereof and a pharmaceutically acceptable excipient.

### DETAILED DESCRIPTION OF THE INVENTION

### Terms and Definitions

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different. For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a saturated straight- or branched-chain hydrocarbon radical having the specified number of carbon atoms. As used herein, the terms “C₁-C₆ alkyl” and “C₁-C₁₀ alkyl” refers to such a group having at least 1 and up to 6 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

The term “C₁-C₁₀-alkenyl” refers to straight or branched hydrocarbon chains containing the specified number of carbon atoms and at least 1, or more, carbon-carbon double bonds. Examples include ethenyl (or ethenylene) and propenyl (or propenylene).

When the term “C₁-C₁₀alkynyl” (or “alkynylene”) is used it refers to straight or branched hydrocarbon chains containing the specified number of carbon atoms and at least 1, or more, carbon-carbon triple bonds. Examples include ethynyl (or ethynylene) and propynyl (or propynylene).

When “C₃-C₆cycloalkyl” is used it refers to a non-aromatic, saturated, cyclic hydrocarbon ring containing the specified number of carbon atoms. So, for example, the term “C₃-C₆ cycloalkyl” refers to a non-aromatic cyclic hydrocarbon ring having from three to eight carbon atoms. Exemplary “C₃-C₆ cycloalkyl” groups useful in the present invention include, but are not limited to, cyclopropyl, cyclobutyl, cyclopentyl, cyclohexyl, cycloheptyl and cyclooctyl.

The term “C₄-C₆cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

“Halo” means the halogen radical fluoro, chloro, bromo, or iodo.

“Haloalkyl” means a C₁-C₁₀alkyl, C₁-C₁₀-alkenyl, or C₁-C₁₀alkynyl group that is substituted with one or more halo substituents. Haloalkyl includes trifluoromethyl.

Where “C₃-C₆ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl and the like.

“Heteroaromatic ring” means an optionally substituted aromatic monocyclic ring or polycarbocyclic fused ring system wherein at least one ring complies with Hückel's Rule, has the specified number of ring atoms, and that ring contains at least one heteratom selected from N, O, and/or S. Examples of these groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” means products whose enantiomeric excess is 99% ee or greater.

“Halo” means the halogen radical fluoro, chloro, bromo, or iodo.

“Haloalkyl” means an alkyl group that is substituted with one or more halo substituents. Haloalkyl includes trifluoromethyl and trifluoroethyl.

Certain compounds of formula (I) have a basic nitrogen group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Certain other compounds of formula (I) have an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

The compounds according to formula (I) may contain one or more asymmetric center, also referred to as a chiral center, and may, therefore, exist as individual enantiomers, diastereomers, or other stereoisomeric forms, or as mixtures thereof. Chiral centers may also be present in a substituent such as an alkyl group. Where the stereochemistry of a chiral center present in formula I, or in any chemical structure illustrated herein, is not specified the structure is intended to encompass any stereoisomer and all mixtures thereof. Thus, compounds according to formula I containing one or more chiral center may be used as racemic mixtures, enantiomerically enriched mixtures, or as enantiomerically pure individual stereoisomers.

Individual stereoisomers of a compound according to formula I which contain one or more asymmetric center may be resolved by methods known to those skilled in the art. For example, such resolution may be carried out (1) by formation of diastereoisomeric salts, complexes or other derivatives; (2) by selective mixture with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing vaiable amounts of water. The invention includes all such solvates.

The skilled artisan will further appreciate that certain compounds of the invention that exist in crystalline form, including the various solvates thereof, may exhibit polymorphism, i.e. the capacity to occur in different crystalline structures. These different crystalline forms are typically known as polymorphs. The invention includes all such polymorphs. Polymorphs typically exhibit different melting points, IR spectra, and X-ray powder diffraction patterns, which may be used for identification. The skilled artisan will appreciate that different polymorphs may be produced, for example, by changing or adjusting the mixture conditions or reagents, used in making the compound. For example, changes in temperature, pressure, or solvent may result in polymorphs. In addition, one polymorph may spontaneously convert to another polymorph under certain conditions.

The compounds of the invention inhibit the cathepsin C enzyme and can be useful in the treatment of conditions wherein the underlying pathology is (at least in part) attributable to cathepsin C involvement or in conditions wherein cathepsin C inhibition offers some clinical benefit even though the underlying pathology is not (even in part) attributable to cathepsin C involvement. Examples of such conditions include COPD, rheumatoid arthritis, osteoarthritis, asthma, and multiple sclerosis. Accordingly, in another aspect the invention is directed to methods of treating such conditions.

The methods of treatment of the invention comprise administering a safe and effective amount of a compound of the invention to a patient in need thereof.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” and “therapeutically effective amount” in reference to a compound of formula I, or a pharmaceutically acceptable salt of it, means an amount of sufficient to significantly induce a positive modification in the condition to be treated but low enough to avoid serious side effects at a reasonable benefit/risk ratio within the scope of sound medical judgment. A safe and effective amount of a compound of the invention will vary with the particular compound chosen, e.g. consider the potency, efficacy, and half-life of the compound; the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 10 mg to 1000 mg.

### Compositions

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein a safe and effective amount of a compound of the invention can be extracted and then given to the patient such as with powders, syrups, and solutions for injection. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically-acceptable excipient. However, in certain embodiments, the pharmaceutical compositions of the invention contain one pharmaceutically-acceptable excipient.

As used herein, “pharmaceutically-acceptable excipient” means a material, composition or vehicle involved in giving form or consistency to the composition and which is safe when administered to a patient. Each excipient must be compatible with the other ingredients of the pharmaceutical composition when comingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compounds of the invention and the pharmaceutically-acceptable excipient or excepients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In another aspect, the invention is directed to a dosage form adapted for administration to a patient by inhalation. For example, the compound of the invention may be inhaled into the lungs as a dry powder, an aerosol, a suspension, or a solution.

Dry powder compositions for delivery to the lung by inhalation typically comprise a compound of the invention as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides.

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of the invention.

Aerosols may be formed by suspending or dissolving a compound of the invention in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of the invention will typically be administered to a patient via a metered dose inhaler (MDI). Such devices are known to those skilled in the art.

The aerosol may contain additional pharmaceutically-acceptable excipients typically used with multiple dose inhalers such as surfactants, lubricants, cosolvents and other excipients to improve the physical stability of the formulation, to improve valve performance, to improve solubility, or to improve taste.

Suspensions and solutions comprising a compound of the invention may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of the invention may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof, or an antioxidant such as antioxidant such as vitamin E or ascorbic acid. These may be used alone or together to stabilize the compound of the invention. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

### ABBREVIATIONS

aq. Aqueous
atm atmosphere
BOC tert-butyloxycarbonyl
DCM/CH₂Cl₂ Dichloromethane
DEAD Diethylazodicarboxylate
DMAP Dimethylaminopyridine
DIPEA/DIEA Di-isopropylethylamine
DMF Dimethylformamide
DPPA Diphenylphosphoryl azide
EA/EtOAc Ethyl acetate
ESI Electrospray ionization
eq. Equivalent
HPLC High pressure liquid chromatography
LC-MS Liquid chromatography-Mass spectrometry
MDAP Mass Directed Auto Prep
Me Methyl
min Minute(s)
ml/mL milliliter
Ph Phenyl
PS Polymer-supported
rt Room temperature
sat or sat. Saturated
SPE Solid phase extraction
TBAF tetra-Butylammonium fluororide
TBS t-Butyldimethyl silyl
TBS-Cl t-Butyldimethyl silyl chloride
TEA Triethylamine
TFA Trifluoroacetic acid
THF Tetrahydrofuran
UV Ultraviolet

### Chemistry Background:

The compounds of this invention may be made by a variety of methods, including those illustrated below. Any previously defined variable will continue to have the previously defined meaning unless otherwise indicated. Illustrative general synthetic methods are set out below and then specific compounds of the invention as prepared are given in the examples.

### Methods of Preparation

The compounds of formula (I) may be obtained by using synthetic procedures illustrated in the Schemes below or by drawing on the knowledge of a skilled organic chemist. The synthesis provided in these Schemes are applicable for producing compounds of the invention having a variety of different R₁ groups employing appropriate precursors, which are suitable protected if needs be, to achieve compatibility with the reactions outlined herein. Subsequent deprotection, where needs be, and then affords compounds of the nature generally disclosed. While the Schemes are shown with compounds only of formula (I), they are illustrative of processes that may be used to make the compounds of the invention.

Compounds names were generated using the software naming program ACD/Name Pro V6.02 available from Advanced Chemistry Development, Inc., 110 Yonge Street, 14^{th} Floor, Toronto, Ontario, Canada, M5C 1T4 (http://www.acdlabs.com/).

As shown in Scheme 1, the compounds of formula (I) can be prepared in a multi-step sequence from the commercially available 1,1-dimethylethyl (3R)-3-amino-1-pyrrolidinecarboxylate 1. Treatment of 1 with a suitable sulfonyl chloride R1SO₂Cl gives an intermediate 2, which can be treated with an alkyl bromide RₓX in a presence of a base such as K₂CO₃ or NaH to give the N-alkylated derivative 3. Subsequent removal of the BOC protecting group of 3 with an acid reagent such as 4N HCl in dioxane followed by treatment with cyanogen bromide results in the formation of the desired compounds 4 of formula (I).

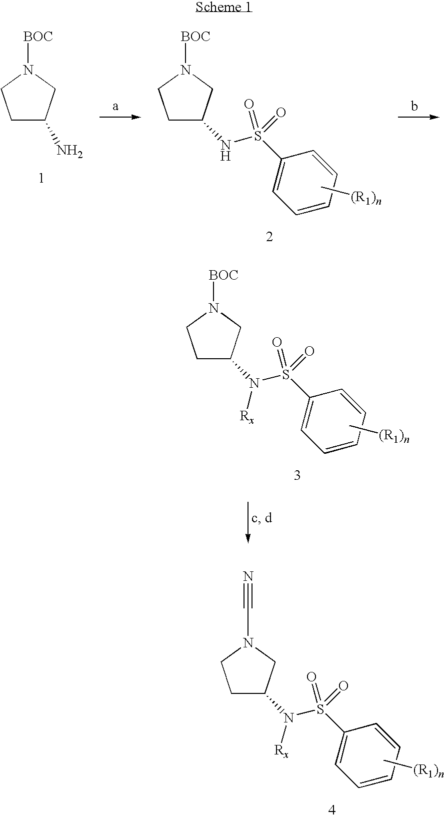

Reagents and conditions: a) (R₁)ₙC₆H₄SO₂Cl, TEA, DCM; b) RₓX, NaH or K₂CO₃; c) 4N HCl, Dioxane; d) BrCN, DIEA, DCM.

### SYNTHETIC EXAMPLES

The invention will now be described by reference to the following Examples which are merely illustrative and are not to be construed as a limitation of the scope of the present invention. All temperatures are given in ° C.

Purifications and analyses of materials were carried out using the following instruments:

### LC-MS Analysis:

Liquid Chromatograph:
System: Shimadzu LC system with SCL-10A Controller and dual UV detector

Autosampler: Leap CTC with a Valco six port injector

Column: Aquasil/Aquasil (C18 40×1 mm)

Inj. Volume (μL): 2.0

Solvent A: H₂O, 0.02% TFA

Solvent B: MeCN, 0.018% TFA

Gradient: linear

Channel A: UV 214 nm

Channel B: ELS

| Step | Time (min) | Dura. (min) | Flow (μL/min) | Sol. A | Sol. B |
|---|---|---|---|---|---|
| 0 | 0.00 | 0.00 | 300.00 | 95.00 | 5.00 |
| 1 | 0.00 | 0.01 | 300.00 | 95.00 | 5.00 |
| 2 | 0.01 | 3.20 | 300.00 | 10.00 | 90.00 |
| 3 | 3.21 | 1.00 | 300.00 | 10.00 | 90.00 |
| 4 | 4.21 | 0.10 | 300.00 | 95.00 | 5.00 |
| 5 | 4.31 | 0.40 | 300.00 | 95.00 | 5.00 |

Mass Spectrometer: PE Sciex Single Quadrupole LC/MS API-150

Polarity: Positive

Acquisition mode: Profile

### Preparatory HPLC

Automated preparatory HPLC purifications were conducted with a Gilson® semi-preparative HPLC system under the following conditions:
Column: 75×33 mm I. D., S-5 um, 12 nm
Flow rate: 30 mL/min
Injection Volume: 0.800 mL
Room temperature
The eluent was a mixture composed of solvents A and B:
   Solvent A: 0.1% trifluoroacetic acid in water
   Solvent B: 0.1% trifluoroacetic acid in acetonitrile

### Automated Flash Chromatography

The automated flash chromatography purifications were conducted with a CombiFlash® Companion® personal flash chromatography system under the following conditions:

Silica cartridge:
Size, 4, 12, 40, 80 or 120 g depending on the amount of material to be purified

Flow rate: Between 4 and 85 mL/min

Room temperature

The eluent was a mixture composed of solvents A and B:
Solvent A: Hexane
Solvent B: Ethyl acetate.

### Reagents:

Unless specified otherwise, the following abbreviations were used for frequently used reagents:

BrCN: A 3 N BrCN solution DCM (Aldrich®, cat #:341894)

PS-trisamine: A tris-(2-aminoethyl)amine polystyrene resin (Argonaut®, p/n 800230)

4 N HCl: A 4 N HCl solution in 1,4-dioxane (Aldrich cat #345547).

### Solvents:

All solvents used herein are of the highest available purity and all reactions are run under anhydrous conditions under an air atmosphere unless otherwise indicated.

### SPECIFIC EXAMPLES

### Intermediate 1 1,1-Dimethylethyl (3R)-3-{[(3-bromophenyl)sulfonyl]amino}-1-pyrrolidinecarboxylate

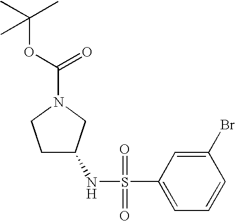

To a chilled (0° C.) solution of 1,1-dimethylethyl (3R)-3-amino-1-pyrrolidinecarboxylate (1 g, 5.37 mmol) and triethylamine (3 ml, 21.5 mmol) in DMF (10 ml) was added 3-bromobenzenesulfonyl chloride (1.51 g, 15.91 mmol). The mixture was stirred at room temperature overnight. The solvent was evaporated. The residue was diluted with DCM, washed with water, and put through a phase separator to dry. The solvent was evaporated to afford the title compound (1.9 g). LC-MS: m/z, 405 (M+H), rt 2.09 min.

### Intermediate 2 1,1-Dimethylethyl (3R)-3-({[2,5-bis(methyloxy)phenyl]sulfonl}amino)-1-pyrrolidinecarboxylate

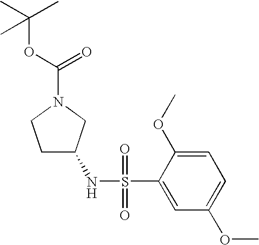

To a chilled (0° C.) solution of 1,1-dimethylethyl (3R)-3-amino-1-pyrrolidinecarboxylate (0.5 ml, 2.95 mmol) and triethylamine (1.60 ml, 11.5 mmol) in DCM (15 ml) was added 2,5-bis(methyloxy)benzenesulfonyl chloride (1.162 g, 3.54 mmol). The mixture was stirred at room temperature overnight. The mixture was diluted with DCM (200 ml), then washed with 0.1M HCl (2×50 ml), sat NaHCO₃ (50 ml), and sat NaCl (50 ml). The organic layer was dried with MgSO4, filtered and concentrated to afford the title compound (1.186 g). LC-MS: m/z, 387 (M+H), rt 0.95 min.

### Intermediate 3, 11-Dimethylethyl (3R)-3-({[5-bromo-2-(methyloxy)phenyl]sulfonyl}amino)-1-pyrrolidinecarboxylate

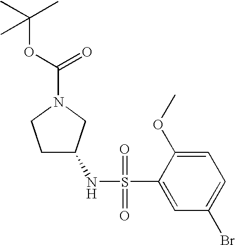

To a chilled (0° C.) solution of 1,1-dimethylethyl (3R)-3-amino-1-pyrrolidinecarboxylate (0.5 ml, 2.95 mmol) and triethylamine (1.60 ml, 11.5 mmol) in DCM (15 ml) was added 5-bromo-2-(methyloxy)benzenesulfonyl chloride (1.0231 g, 3.58 mmol). The mixture was stirred at room temperature overnight. The mixture was diluted with DCM (200 ml), then washed with 0.1M HCl (2×50 ml), sat NaHCO₃ (50 ml), and sat NaCl (50 ml). The organic layer was dried over MgSO₄, filtered and concentrated to afford the title compound (1.2857 g). LC-MS: m/z, 435 (M+H), rt 1.05 min.

### Intermediate 4 1,1-dimethylethyl (3R)-3-{[(2,5-dichlorophenyl)sulfonyl]amino}-1-pyrrolidinecarboxylate

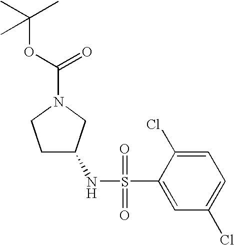

To a chilled (0° C.) solution of 1,1-dimethylethyl (3R)-3-amino-1-pyrrolidinecarboxylate (1.4 ml, 8.25 mmol) and triethylamine (3.5 ml, 25.1 mmol) in DCM (50 ml) was added 2,5-dichlorobenzenesulfonyl chloride (2.229 g, 9.08 mmol). The mixture was stirred at room temperature overnight. The mixture was diluted with DCM (300 ml), then washed with 0.1M HCl (2×100 ml), sat NaHCO₃ (100 ml), and sat NaCl (100 ml). The organic layer was dried over MgSO₄, filtered and concentrated to afford the title compound (2.9971 g). LC-MS: m/z, 395 (M+H), rt 2.09 min.

### Intermediate 5, 11-Dimethylethyl (3R)-3-({[2-chloro-5-(trifluoromethyl)phenyl]sulfonyl}amino)-1-pyrrolidinecarboxylate

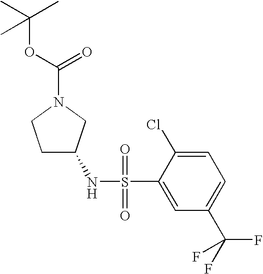

To a chilled (0° C.) solution of 1,1-dimethylethyl (3R)-3-amino-1-pyrrolidinecarboxylate (0.25 ml, 1.474 mmol) and triethylamine (0.62 ml, 4.45 mmol) in DCM (10 ml) was added 2-chloro-5-(trifluoromethyl)benzenesulfonyl chloride (0.5003 g, 1.793 mmol). The mixture was stirred at room temperature overnight. The mixture was diluted with DCM (200 ml), then washed with 0.1M HCl (2×50 ml), sat NaHCO₃ (50 ml), and sat NaCl (50 ml). The organic layer was dried over MgSO₄, filtered and concentrated to afford the title compound (0.5992 g). LC-MS: m/z, 429 (M+H), rt 1.13 min.

### Intermediate 6 1,1-dimethylethyl (3R)-3-{[(2,5-dibromophenyl)sulfonyl]amino}-1-pyrrolidinecarboxylate

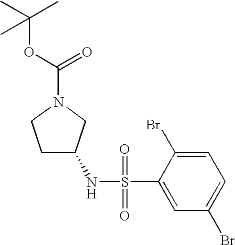

To a chilled (0° C.) solution of 1,1-dimethylethyl (3R)-3-amino-1-pyrrolidinecarboxylate (0.911 ml, 5.37 mmol) and triethylamine (2.25 ml, 16.14 mmol) in DCM (30 ml) was added 2,5-dibromobenzenesulfonyl chloride (1.7939 g, 5.36 mmol). The mixture was stirred at room temperature overnight. The mixture was diluted with DCM (350 ml), then washed with 0.1M HCl (2×100 ml), sat NaHCO₃ (100 ml), and sat NaCl (100 ml). The organic layer was dried over MgSO4, filtered and concentrated to afford the title compound (2.4674 g). LC-MS: m/z, 484 (M+H), rt 2.15 min.

### Intermediate 7 1,1-dimethylethyl(3R)-3-[({2,5-bis[(2,2,2-trifluoroethyl)oxy]phenyl}sulfonyl)amino]-1-pyrrolidinecarboxylate

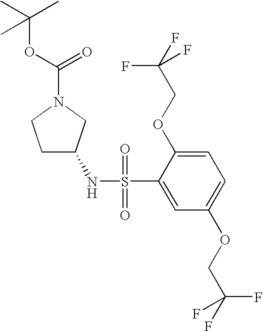

To a chilled (0° C.) solution of 1,1-dimethylethyl (3R)-3-amino-1-pyrrolidinecarboxylate (0.18 ml, 1.061 mmol) and triethylamine (0.42 ml, 3.01 mmol) in DCM (4 ml) was added 2,5-bis[(2,2,2-trifluoroethyl)oxy]benzenesulfonyl chloride (0.4106 g, 1.102 mmol). The mixture was stirred at room temperature overnight. The mixture was diluted with DCM (100 ml), then washed with 0.1M HCl (2×50 ml), sat NaHCO3 (50 ml), and sat NaCl (50 ml). The organic layer was dried MgSO₄, filtered and concentrated to afford the title compound (0.4584 g). LC-MS: m/z, 523 (M+H), rt 1.17 min.

### Intermediate 8 1,1-dimethylethyl(3R)-3-[({2-(methyloxy)-5-[(methyloxy)carbonyl]phenyl}sulfonyl)amino]-1-pyrrolidinecarboxylate

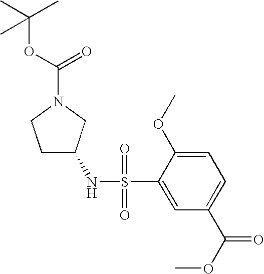

To a chilled (0° C.) solution of 1,1-dimethylethyl (3R)-3-amino-1-pyrrolidinecarboxylate (0.18 ml, 1.061 mmol) and triethylamine (0.42 ml, 3.01 mmol) in DCM (4 ml) was added methyl 3-(chlorosulfonyl)-4-(methyloxy)benzoate (0.309 g, 1.167 mmol). The mixture was stirred at room temperature overnight. The mixture was diluted with DCM (100 ml), then washed with 0.1M HCl (2×50 ml), sat NaHCO3 (50 ml), and sat NaCl (50 ml). The organic layer was dried MgSO₄, filtered and concentrated to afford the title compound (0.3468 g). LC-MS: m/z, 415 (M+H), rt 1.01 min.

### Example 1

### 3-Bromo-N-[(3R)-1-cyano-3-pyrrolidinyl]-N-methylbenzenesulfonamide

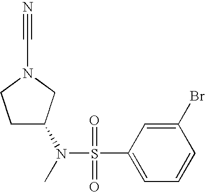

To a solution of 1,1-dimethylethyl (3R)-3-{[(3-bromophenyl)sulfonyl]amino}-pyrrolidinecarboxylate (0.257 g, 0.634 mmol) in acetone (5 ml) was added K₂CO₃ (0.113 g, 0.817 mmol) and bromomethane (0.388 ml, 2.0 M in diethyl ether, 0.776 mmol). The mixture was stirred at room temperature overnight. The mixture was filtered, and the solvent was evaporated. To the residue was added DCM (5 ml) and 4N HCl in 1,4-dioxane (4.0 ml). After stirring at room temperature overnight, the solvent was evaporated. The residue was diluted with DCM (20 ml), and mixed with DIEA (0.3 mL, 1.72 mmol) and BrCN (0.60 mL, 1.8 mmol). The resultant mixture was stirred at room temperature overnight. The solvent was evaporated and the residue purified by preparatory HPLC (without TFA) to afford the title compound (0.1377 g). LC-MS: m/z, 344 (M+H), rt 1.80 min.

### Example 2

### 3-Bromo-N-[(3R)-1-cyano-3-pyrrolidinyl]-N-(phenylmethyl)benzenesulfonamide

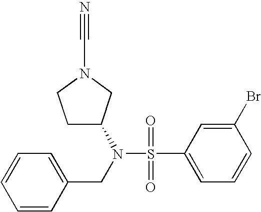

To a solution of 1,1-dimethylethyl (3R)-3-{[(3-bromophenyl)sulfonyl]amino}-pyrrolidinecarboxylate (0.257 g, 0.634 mmol) in acetone (5 ml) was added K₂CO₃ (0.111 g, 0.817 mmol) and (bromomethyl)benzene (0.093 ml, 0.782 mmol). The mixture was stirred at room temperature overnight. The mixture was filtered, and the solvent was evaporated. To the residue was added DCM (5 ml) and 4N HCl in 1,4-dioxane (4.0 ml). After stirring at room temperature overnight, the solvent was evaporated. The residue was diluted with DCM (20 ml), and mixed with DIEA (0.3 mL, 1.72 mmol) and BrCN (0.60 mL, 1.8 mmol). The resultant mixture was stirred at room temperature overnight. The solvent was evaporated and the residue purified by preparatory HPLC (without TFA) to afford the title compound (0.1511 g). LC-MS: m/z, 420 (M+H), rt 2.20 min.

### Example 3

### N-[(3R)-1-Cyano-3-pyrrolidinyl]-N-methyl-2,5-bis(methyloxy)benzenesulfonamide

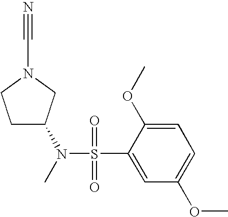

To a solution of 1,1-dimethylethyl (3R)-3-({[2,5-bis(methyloxy)phenyl]sulfonyl}amino)-1-pyrrolidinecarboxylate (0.390 g, 1.01 mmol) in acetone was added K₂CO₃ (0.140 g, 1.01 mmol) and bromomethane (0.53 ml, 2.0 M in ether, 1.06 mmol). The mixture was stirred at room temperature overnight, then heated at 45° C. for 2 days. The mixture was cooled to room temperature and PPh₃ polymer bound resin (0.333 g, 3 mmol/g, 1.00 mmol) was added. The mixture was then filtered through celite and the solvents evaporated. To the residue was added DCM (5 ml) and 4N HCl in 1,4-dioxane (4 ml). After stirring at room temperature, the solvent was evaporated. The mixture was diluted with DCM (10 ml), and mixed with DIEA (0.7 mL, 4.02 mmol) and BrCN (0.70 mL, 2.10 mmol). The resultant mixture was stirred at room temperature overnight. PS-trisamine resin (1.05 g) was added to the mixture for 2 hours, then the mixture was filtered, concentrated and the concentrate purified by preparatory HPLC (without TFA) to afford the title compound (0.0205 g). LC-MS: m/z, 326 (M+H), rt 1.53 min.

### Example 4

### N-[(3R)-1-Cyano-3-pyrrolidinyl]-2,5-bis(methyloxy)-N-(phenylmethyl)benzenesulfonamide

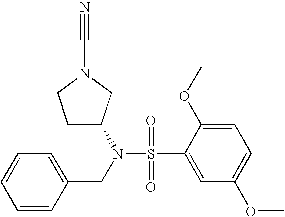

To a solution of 1,1-dimethylethyl (3R)-3-({[2,5-bis(methyloxy)phenyl]sulfonyl}amino)-1-pyrrolidinecarboxylate (0.390 g, 1.01 mmol) in acetone was added K₂CO₃ (0.140 g, 1.01 mmol) and benzyl bromide (0.13 ml, 1.09 mmol). The mixture was stirred at room temperature overnight, and then heated at 45° C. for 2 days. The mixture was cooled to room temperature and PPh₃ polymer bound resin (0.333 g, 3 mmol/g, 1.00 mmol) was added. The mixture was then filtered through celite and the solvents evaporated. To the residue was added DCM (5 ml) and 4N HCl in 1,4-dioxane (4 ml). After stirring at room temperature, the solvent was evaporated. The residue was diluted with DCM (10 ml), and mixed with DIEA (0.7 mL, 4.02 mmol) and BrCN (0.70 mL, 2.10 mmol). The resultant mixture was stirred at room temperature overnight. PS-trisamine resin (1.05 g) was added for 2 hours, then the mixture was filtered, concentrated and the concentrate purified by preparatory HPLC (without TFA) to afford the title compound (0.0698 g). LC-MS: m/z, 402 (M+H), rt 1.96 min.

### Example 5

### 5-Bromo-N-[(3R)-1-cyano-3-pyrrolidinyl]-N-methyl-2-(methyloxy)benzenesulfonamide

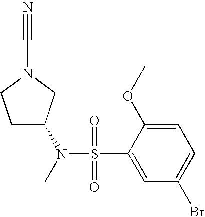

To a solution of 1,1-dimethylethyl (3R)-3-({[5-bromo-2-(methyloxy)phenyl]sulfonyl}amino)-1-pyrrolidinecarboxylate (0.400 g, 0.919 mmol) in acetone was added K₂CO₃ (0.140 g, 1.01 mmol) and bromomethane (0.48 ml, 2.0 M in ether, 0.96 mmol). The mixture was stirred at room temperature overnight, and then heated at 45° C. for overnight. The mixture was cooled to room temperature and PPh₃ polymer bound resin (0.333 g, 3 mmol/g, 1.00 mmol) was added. The mixture was then filtered through celite and the solvents evaporated. To the residue was added DCM (10 ml) and 4N HCl in 1,4-dioxane (5 ml). After stirring at room temperature, the solvent was evaporated. The resulting residue was diluted with DCM, and mixed with DIEA (0.6 mL, 3.44 mmol) and BrCN (0.28 mL, 1.74 mmol). The mixture was stirred at room temperature overnight. PS-trisamine resin (1.05 g) was added for over the weekend, then the mixture was filtered, concentrated and the concentrate purified by preparatory HPLC (without TFA) to afford the title compound (0.0582 g). LC-MS: m/z, 374 (M+H), rt 1.76 min.

### Example 6

### 5-Bromo-N-[(3R)-1-cyano-3-pyrrolidinyl]-2-(methyloxy)-N-(phenylmethyl)benzenesulfonamide

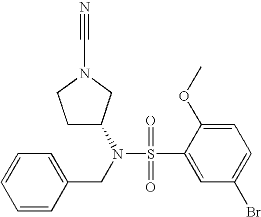

To a solution of 1,1-dimethylethyl (3R)-3-({[5-bromo-2-(methyloxy)phenyl]sulfonyl}amino)-1-pyrrolidinecarboxylate (0.400 g, 0.919 mmol) in acetone was added K₂CO₃ (0.140 g, 1.01 mmol) and benzyl bromide (0.12 ml, 1.01 mmol). The mixture was stirred at room temperature overnight, then heated at 45° C. for overnight. The mixture was cooled to room temperature and PPh₃ polymer bound resin (0.333 g, 3 mmol/g, 1.00 mmol) was added to the mixture. The mixture was filtered through celite and the solvents evaporated. To the residue was added DCM (10 ml) and 4N HCl in 1,4-dioxane (5 ml). After stirring at room temperature, the solvent was evaporated. The residue was diluted with DCM, and mixed with DIEA (0.6 mL, 3.44 mmol) and BrCN (0.28 mL, 1.74 mmol). The mixture was stirred at room temperature overnight. PS-trisamine resin (1.05 g) was added for over the weekend, then the mixture was filtered, concentrated and the concentrate purified by preparatory HPLC (without TFA) to afford the title compound (0.0449 g). LC-MS: m/z, 450 (M+H), rt 2.13 min.

### Example 7

### 2,5-Dichloro-N-[(3R)-1-cyano-3-pyrrolidinyl]-N-methylbenzenesulfonamide

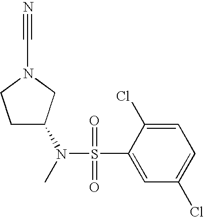

To a solution of 1,1-dimethylethyl (3R)-3-{[(2,5-dichlorophenyl)sulfonyl]amino}-1-pyrrolidinecarboxylate (0.398 g, 1.01 mmol) in acetone was added K₂CO₃ (0.153 g, 1.12 mmol) and bromomethane (0.53 ml, 2.0 M in ether, 1.06 mmol). The mixture was stirred at room temperature overnight, then heated at 45° C. for 2 days. It was cooled to room temperature and PPh₃ polymer bound resin (0.333 g, 3 mmol/g, 1.00 mmol) was added. This mixture was then filtered through celite and the solvents evaporated. To the residue was added DCM (10 ml) and 4N HCl in 1,4-dioxane (5 ml). After stirring at room temperature, the solvent was evaporated. The residue was diluted with DCM, and mixed with DIEA (0.77 mL, 4.42 mmol) and BrCN (0.74 mL, 2.22 mmol). The resulting mixture was stirred at room temperature overnight. PS-trisamine resin was added for over the weekend, then the mixture was filtered, concentrated and purified by preparatory HPLC (without TFA) to afford the title compound (0.0659 g). LC-MS: m/z, 336 (M+H), rt 1.84 min.

### Example 8

### 2,5-dichloro-N-[(3R)-1-cyano-3-pyrrolidinyl]-N-(phenylmethyl)benzenesulfonamide

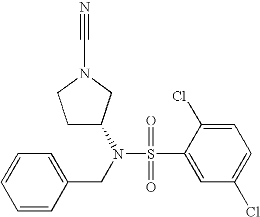

To a solution of 1,1-dimethylethyl (3R)-3-{[(2,5-dichlorophenyl)sulfonyl]amino}-1-pyrrolidinecarboxylate (0.398 g, 1.01 mmol) in acetone was added K₂CO₃ (0.153 g, 1.12 mmol) and benzyl bromide (0.12 ml, 1.01 mmol). The mixture was stirred at room temperature overnight, then heated at 45° C. for overnight. It was cooled to room temperature and PPh₃ polymer bound resin (0.333 g, 3 mmol/g, 1.00 mmol) was added. The mixture was then filtered through celite and the solvents evaporated. To the residue was added DCM (10 ml) and 4N HCl in 1,4-dioxane (5 ml). After stirring at room temperature, the solvent was evaporated. The residue was diluted with DCM, and mixed with DIEA (0.77 mL, 4.42 mmol) and BrCN (0.74 mL, 2.22 mmol). The mixture was stirred at room temperature overnight. PS-trisamine resin (1.05 g) was added to the mixture for over the weekend, then it was filtered, concentrated and purified by preparatory HPLC (without TFA) to afford the title compound (0.1219 g). LC-MS: m/z, 410 (M+H), rt 2.24 min.

### Example 9

### 2-Chloro-N-[(3R)-1-cyano-3-pyrrolidinyl]-N-methyl-5-(trifluoromethyl)benzenesulfonamide

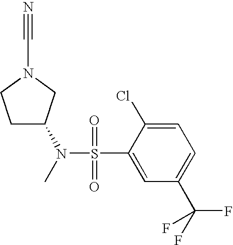

To a solution of 1,1-dimethylethyl (3R)-3-({[2-chloro-5-(trifluoromethyl)phenyl]sulfonyl}amino)-1-pyrrolidinecarboxylate (0.415 g, 0.968 mmol) in acetone was added K₂CO₃ (0.147 g, 1.06 mmol) and bromomethane (0.51 ml, 2.0 M in Ether, 1.02 mmol). The reaction mixture was stirred at room temperature overnight, then heated at 45° C. for 2 days. The mixture was cooled to room temperature and PPh₃ polymer bound resin (0.333 g, 3 mmol/g, 1.00 mmol) was added to the mixture. The mixture was then filtered through celite and the solvents evaporated. To the residue was added DCM (5 ml) and 4N HCl in 1,4-dioxane (5 ml). After stirring at room temperature, the solvent was evaporated. The residue was diluted with DCM, and mixed with DIEA (0.77 mL, 4.42 mmol) and BrCN (0.74 mL, 2.22 mmol). The resultant mixture was stirred at room temperature overnight. PS-trisamine resin was added to the mixture for over the weekend, then the mixture was filtered, concentrated and purified by preparatory HPLC (without TFA) to afford the title compound (0.040 g). LC-MS: m/z, 368 (M+H), rt 1.93 min.

### Example 10

### 2-Chloro-N-[(3R)-1-cyano-3-pyrrolidinyl]-N-(phenylmethyl)-5-(trifluoromethyl)benzenesulfonamide

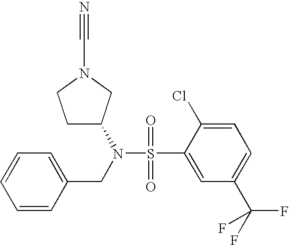

To a solution of 1,1-dimethylethyl (3R)-3-({[2-chloro-5-(trifluoromethyl)phenyl]sulfonyl}amino)-1-pyrrolidinecarboxylate (0.415 g, 0.968 mmol) in acetone was added K₂CO₃ (0.147 g, 1.06 mmol) and benzyl bromide (0.12 ml, 1.01 mmol). The mixture was stirred at room temperature overnight, then heated at 45° C. for 2 days. It was cooled to room temperature and PPh₃ polymer bound resin (0.333 g, 3 mmol g, 1.00 mmol) was added to the mixture. It was then filtered through celite and the solvents evaporated. To the residue was added DCM (5 ml) and 4N HCl in 1,4-dioxane (5 ml). After stirring at room temperature, the solvent was evaporated. The residue was diluted with DCM, and mixed with DIEA (0.77 mL, 4.42 mmol) and BrCN (0.74 mL, 2.22 mmol). The resultant mixture was stirred at room temperature overnight. PS-trisamine resin (1.05 g) was added to the mixture over the weekend, then it was filtered, concentrated and purified by preparatory HPLC (without TFA) to afford the title compound (0.130 g). LC-MS: m/z, 444 (M+H), rt 2.28 min.

### Example 11

### 2,5-Dibromo-N-[(3R)-1-cyano-3-ppyrrolidinyl]-N-methylbenzenesulfonamide

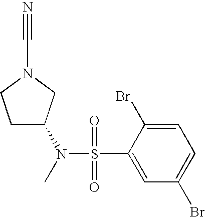

To a solution of 1,1-dimethylethyl (3R)-3-{[(2,5-dibromophenyl)sulfonyl]amino}-1-pyrrolidinecarboxylate (0.400 g, 0.826 mmol) in acetone was added K₂CO₃ (0.124 g, 0.897 mmol) and bromomethane (0.45 ml, 2.0 M in ether, 0.90 mmol). The mixture was stirred at room temperature over the weekend. PPh₃ polymer bound resin (3 mmol/g) was added. The mixture was then filtered and concentrated. To the residue was added DCM and 4N HCl in 1,4-dioxane (5 ml). After stirring at room temperature overnight, the solvent was evaporated. The residue was diluted with DCM, and mixed with DIEA (0.6 mL, 3.44 mmol) and BrCN (0.55 mL, 1.65 mmol). The resultant material was stirred at room temperature overnight. PS-trisamine resin was added for 3 hours, then the mixture was filtered, concentrated and purified by preparatory HPLC (without TFA) to afford the title compound (0.0826 g). LC-MS: m/z, 422 (M+H), rt 1.92 min.

### Example 12

### 2,5-Dibromo-N-[(3R)-1-cyano-3-pyrrolidinyl]-N-(phenylmethyl)benzenesulfonamide

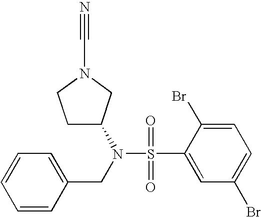

To a solution of 1,1-dimethylethyl (3R)-3-{[(2,5-dibromophenyl)sulfonyl]amino}-1-pyrrolidinecarboxylate (0.400 g, 0.826 mmol) in acetone was added K₂CO₃ (0.124 g, 0.897 mmol) and benzyl bromide (0.12 ml, 1.01 mmol). The mixture was stirred at room temperature over the weekend. PPh₃ polymer bound resin (3 mmol/g) was added to the mixture. It was then filtered and concentrated. To the residue was added DCM and 4N HCl in 1,4-dioxane (5 ml). After stirring at room temperature overnight, the solvent was evaporated. The residue was diluted with DCM, and mixed with DIEA (0.6 mL, 3.44 mmol) and BrCN (0.55 mL, 1.65 mmol). The resultant mixture was stirred at room temperature overnight. PS-trisamine resin was added to the mixture for 3 hours, then it was filtered, concentrated and purified by preparatory HPLC (without TFA) to afford the title compound (0.1825 g). LC-MS: m/z, 498 (M+H), rt 2.32 min.

### Example 13

### 2,5-Dichloro-N-[(3R)-1-cyano-3-pyrrolidinyl]-N-propylbenzenesulfonamide

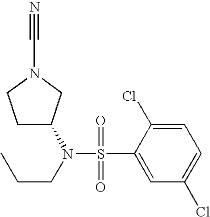

To a chilled (0° C.) solution of 1,1-dimethylethyl (3R)-3-{[(2,5-dichlorophenyl)sulfonyl]amino}-1-pyrrolidinecarboxylate (0.1013 g, 0.256 mmol) in DMF (2 ml) was added NaH (0.046 g, 1.821 mmol, 95%). The mixture was stirred for 5 minutes, and then 1-bromopropane (0.13 ml, 1.431 mmol) was added. It was stirred at room temperature overnight, then heated at 55° C. for overnight. The mixture was cooled to room temperature and diluted with EtOAc (25 ml). The organic layer was washed with water (2×20 ml), dried MgSO₄, filtered and concentrated. The residue was purified by automated flash chromatography (12 g silica cartridge) to afford 1,1-dimethylethyl (3R)-3-[[(2,5-dichlorophenyl)sulfonyl](propyl)amino]-1-pyrrolidinecarboxylate (0.036 g, 0.082 mmol). LC-MS: m/z, 437 (M+H), rt 1.29 min.

To this compound was added DCM (2 ml) and 4N HCl in 1,4-dioxane (1.0 ml). After stirring at room temperature for 90 min, the solvent was evaporated. The residue was diluted with DCM (3 ml) and DIEA (0.05 mL, 0.286 mmol) and stirred for 10 min before BrCN (0.040 mL, 0.120 mmol) was added. The mixture was stirred at room temperature overnight. PS-trisamine resin (1 equiv) was added to the mixture and stirred for 2 hours, then it was filtered, concentrated and purified by preparatory HPLC (without TFA) to afford the title compound (11.6 mg). LC-MS: m/z, 362 (M+H), rt 1.18 min.

### Example 14

### N-[(3R)-1-Cyano-3-pyrrolidinyl]-2,5-bis(methyloxy)-N-propylbenzenesulfonamide

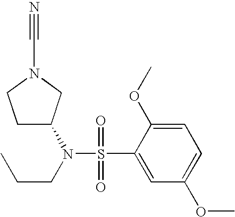

To a chilled (0° C.) solution of 1,1-dimethylethyl (3R)-3-({[2,5-bis(methyloxy)phenyl]sulfonyl}amino)-1-pyrrolidinecarboxylate (0.2023 g, 0.523 mmol) in DMF (3 ml) was added NaH (0.086 g, 3.4 mmol, 95%). The mixture was stirred for 5 minutes, and then 1-bromopropane (0.24 ml, 2.64 mmol) was added. It was then warmed to room temperature, then heated to 55° C. for overnight. The mixture was cooled to room temperature and diluted with EtOAc (50 ml). The organic layer was washed with water (2×25 ml), dried MgSO₄, filtered and concentrated. The residue was purified by automated flash chromatography (40 g silica cartridge, Hex\EA) to afford 1,1-dimethylethyl (3R)-3-[{[2,5-bis(methyloxy)phenyl]sulfonyl}(propyl)amino]-1-pyrrolidinecarboxylate (0.1896 g, 0.442 mmol). LC-MS: m/z, 429 (M+H), rt 1.15 min.

To this compound was added DCM (2 ml) and 4N HCl in 1,4-dioxane (2.0 ml, 8.00 mmol). After stirring at room temperature for 90 min, the solvent was evaporated. The residue was diluted with DCM (3 ml) and DIEA (0.23 mL, 1.317 mmol) and stirred for 10 min before BrCN (0.22 mL, 0.660 mmol) was added. The mixture was stirred at room temperature overnight. PS-trisamine resin (1 equiv) was added to the mixture and stirred for 2 hours, then it was filtered, concentrated and purified by preparatory HPLC (without TFA) to afford the title compound (0.0893 g). LC-MS: m/z, 354 (M+H), rt 0.95 min.

### Example 15

### 2,5-Dibromo-N-[(3R)-1-cyano-3-pyrrolidinyl]-N-propylbenzenesulfonamide

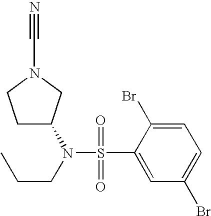

To a chilled (0° C.) solution of 1,1-dimethylethyl (3R)-3-{[(2,5-dibromophenyl)sulfonyl]amino}-1-pyrrolidinecarboxylate (0.1576 g, 0.325 mmol) in DMF (3 ml) was added NaH (0.0446 g, 1.766 mmol, 95%). The mixture was stirred for 5 minutes, and then 1-bromopropane (0.14 ml, 1.541 mmol) was added. The mixture was then warmed to room temperature then heated to 55° C. for overnight. It was cooled to room temperature and diluted with EtOAc (50 ml). The organic layer was washed with water (2×25 ml), dried MgSO₄, filtered and concentrated. The residue was purified by automated flash chromatography (40 g silica cartridge, Hex\EA) to afford 1,1-dimethylethyl (3R)-3-[[(2,5-dibromophenyl)sulfonyl](propyl)amino]-1-pyrrolidinecarboxylate (0.099 g, 0.188 mmol). LC-MS: m/z, 527 (M+H), rt 1.32 min.

To this compound was added DCM (2 ml) and 4N HCl in 1,4-dioxane (1.5 ml, 6.00 mmol). After stirring at room temperature for 90 min, the solvent was evaporated. The residue was diluted with DCM (3 ml) and DIEA (0.1 mL, 0.573 mmol) and stirred for 10 min before BrCN (0.1 mL, 0.300 mmol) was added. The mixture was stirred at room temperature for 2 hours. PS-trisamine resin (1 equiv) was added to the mixture and it was stirred for 2 hours, then it was filtered, concentrated and purified by preparatory HPLC (without TFA) to afford the title compound (0.031 g). LC-MS: m/z, 452 (M+H), rt 1.09 min.

### Example 16

### 2,5-Dibromo-N-[(3R)-1-cyano-3-pyrrolidinyl]-N-(2-methylpropyl)benzenesulfonamide

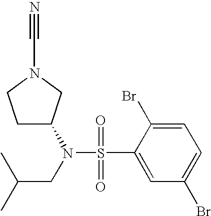

To a chilled (0° C.) solution of 1,1-dimethylethyl (3R)-3-{[(2,5-dibromophenyl)sulfonyl]amino}-1-pyrrolidinecarboxylate (0.1527 g, 0.315 mmol) in DMF (3 ml) was added NaH (0.0456 g, 1.805 mmol, 95%). The mixture was stirred for 5 minutes, and then 1-bromo-2-methylpropane (0.17 ml, 1.563 mmol) was added. The mixture was warmed to room temperature then heated to 55° C. for overnight. The mixture was then heated to 65° C. for over the weekend. The mixture was cooled to room temperature, concentrated, and purified by preparatory HPLC (without TFA) to afford 1,1-dimethylethyl (3R)-3-[[(2,5-dibromophenyl)sulfonyl](2-methylpropyl)amino]-1-pyrrolidinecarboxylate (0.0444 g, 0.082 mmol). LC-MS: m/z, 541 (M+H), rt 1.35 min.

To this compound was added DCM (2 ml) and 4N HCl in 1,4-dioxane (1.0 ml, 4.00 mmol). The mixture was stirred at room temperature overnight, and then the solvent was evaporated. The residue was diluted with DCM (3 ml) and DIEA (0.05 mL, 0.286 mmol) and stirred for 10 min before BrCN (0.05 mL, 0.150 mmol) was added. The mixture was stirred at room temperature for 1 hour. PS-trisamine resin (1 equiv) was added to the mixture and stirred overnight, then the mixture was filtered, concentrated and purified by preparatory HPLC (without TFA) to afford the title compound (0.0204 g). LC-MS: m/z, 466 (M+H), rt 1.12 min.

### Example 17

### 2,5-Dibromo-N-[(3R)-1-cyano-3-pyrrolidinyl]-N-(cyclohexylmethyl)benzenesulfonamide

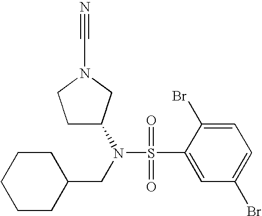

To a chilled (0° C.) solution of 1,1-dimethylethyl (3R)-3-{[(2,5-dibromophenyl)sulfonyl]amino}-1-pyrrolidinecarboxylate (0.15 g, 0.310 mmol) in DMF (3 ml) was added NaH (0.06 g, 2.375 mmol, 95%). The mixture was stirred for 5 minutes, then (bromomethyl)cyclohexane (0.22 ml, 1.577 mmol) was added. The mixture was warmed to room temperature then heated to 65° C. over the weekend. The mixture was cooled to room temperature, concentrated, and purified by preparatory HPLC (without TFA) to afford 1,1-dimethylethyl (3R)-3-{(cyclohexylmethyl)[(2,5-dibromophenyl)sulfonyl]amino}-1-pyrrolidinecarboxylate (0.0746 g, 0.129 mmol). LC-MS: m/z, 581 (M+H), rt 1.45 min. To this compound was added DCM (2 ml) and 4N HCl in 1,4-dioxane (1.0 ml, 4.00 mmol). The mixture was stirred at room temperature for 2 hours, and then the solvent was evaporated. The residue was diluted with DCM (3 ml) and DIEA (0.07 mL, 0.401 mmol) and stirred for 10 min before BrCN (0.07 mL, 0.210 mmol) was added. The mixture was stirred at room temperature for 1 hour. PS-trisamine resin (1 equiv) was added to the mixture and stirred for 2 hours, then the mixture was filtered, concentrated and purified by preparatory HPLC (without TFA) to afford the title compound (0.0116 g). LC-MS: m/z, 506 (M+H), rt 1.28 min.

### Example 18

### 2,5-Dibromo-N-[(3R)-1-cyano-3-pyrrolidinyl]-N-hexylbenzenesulfonamide

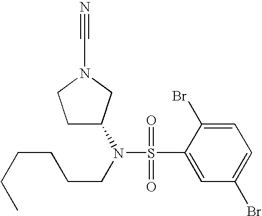

To a chilled (0° C.) solution of 1,1-dimethylethyl (3R)-3-{[(2,5-dibromophenyl)sulfonyl]amino}-1-pyrrolidinecarboxylate (0.15 g, 0.310 mmol) in DMF (3 ml) was added NaH (0.06 g, 2.375 mmol, 95%). The mixture was stirred for 5 minutes, and then 1-bromohexane (0.23 ml, 1.639 mmol) was added. The mixture was warmed to room temperature then heated to 65° C. over the weekend. The mixture was cooled to room temperature, concentrated, and purified by preparatory HPLC (without TFA) to afford 1,1-dimethylethyl (3R)-3-[[(2,5-dibromophenyl)sulfonyl](hexyl)amino]-1-pyrrolidinecarboxylate (0.0449 g, 0.079 mmol). LC-MS: m/z, 569 (M+H), rt 1.44 min. To this compound was added DCM (2 ml) and 4N HCl in 1,4-dioxane (1.0 ml, 4.00 mmol). The mixture was stirred at room temperature for 2 hours, then the solvent was evaporated. The residue was diluted with DCM (3 ml) and DIEA (0.05 mL, 0.286 mmol) and stirred for 10 min before BrCN (0.04 mL, 0.120 mmol) was added. The mixture was stirred at room temperature for 1 hour. PS-trisamine resin (1 equiv) was added to the mixture and stirred for 2 hours, then the mixture was filtered, concentrated and purified by preparatory HPLC (without TFA) to afford the title compound (0.0148 g). LC-MS: m/z, 494 (M+H), rt 1.27 min.

### Example 19

### N-[(3R)-1-Cyano-3-pyrrolidinyl]-2,5-bis(methyloxy)-N-(2-methylpropyl)benzenesulfonamide

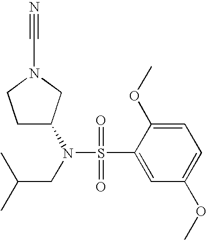

To a chilled (0° C.) solution of 1,1-dimethylethyl (3R)-3-({[2,5-bis(methyloxy)phenyl]sulfonyl}amino)-1-pyrrolidinecarboxylate (0.1553 g, 0.402 mmol) in DMF (3 ml) was added NaH (0.0640 g, 2.53 mmol, 95%). The mixture was stirred for 5 minutes, and then 1-bromo-2-methylpropane (0.21 ml, 1.931 mmol) was added. The mixture was warmed to room temperature then heated to 55° C. overnight. The mixture was then heated to 65° C. over the weekend. The mixture was cooled to room temperature, concentrated, and purified by preparatory HPLC (without TFA) to afford 1,1-dimethylethyl (3R)-3-[{[2,5-bis(methyloxy)phenyl]sulfonyl}(2-methylpropyl)amino]-1-pyrrolidinecarboxylate (0.0543 g, 0.123 mmol). LC-MS: m/z, 443 (M+H), rt 1.17 min. To this compound was added DCM (2 ml) and 4N HCl in 1,4-dioxane (1.0 ml, 4.00 mmol). The mixture was stirred at room temperature overnight, then the solvent was evaporated. The residue was diluted with DCM (3 ml) and DIEA (0.07 mL, 0.401 mmol) and stirred for 10 min before BrCN (0.07 mL, 0.210 mmol) was added. The mixture was stirred at room temperature for 1 hour. PS-trisamine resin (1 equiv) was added to the mixture and stirred overnight, then the mixture was filtered, concentrated and purified by preparatory HPLC (without TFA) to afford the title compound (0.0293 g). LC-MS: m/z, 368 (M+H), rt 1.01 min.

### Example 20

### N-[(3R)-1-Cyano-3-pyrrolidinyl]-N-ethyl-2,5-bis(methyloxy)benzenesulfonamide

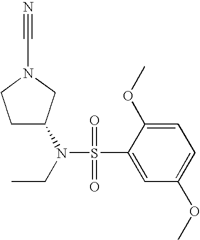

To a chilled (0° C.) solution of 1,1-dimethylethyl (3R)-3-({[2,5-bis(methyloxy)phenyl]sulfonyl}amino)-1-pyrrolidinecarboxylate (0.1760 g, 0.455 mmol) in DMF (3 ml) was added NaH (0.115 g, 4.55 mmol, 95%). The mixture was stirred for 5 minutes, and then bromoethane (0.15 ml, 2.010 mmol) was added. The mixture was warmed to room temperature then heated to 50° C. overnight. The mixture was cooled to room temperature, concentrated, and purified by preparatory HPLC (without TFA) to afford 1,1-dimethylethyl (3R)-3-[{[2,5-bis(methyloxy)phenyl]sulfonyl}(ethyl)amino]-1-pyrrolidinecarboxylate (0.0864 g, 0.208 mmol). LC-MS: m/z, 415 (M+H), rt 1.07 min. To this compound was added DCM (2 ml) and 4N HCl in 1,4-dioxane (1.0 ml, 4.00 mmol). The mixture was stirred at room temperature for 3 hours, then the solvent was evaporated. The residue was diluted with DCM (3 ml) and DIEA (0.11 mL, 0.63 mmol) and stirred for 10 min before BrCN (0.11 mL, 0.330 mmol) was added. The mixture was stirred at room temperature for 1 hour. PS-trisamine resin (1 equiv) was added to the mixture and stirred for 2 hours, then the mixture was filtered, concentrated and purified by preparatory HPLC (without TFA) to afford the title compound (0.0126 g). LC-MS: m/z, 340 (M+H), rt 0.90 min.

### Example 21

### N-[(3R)-1-Cyano-3-pyrrolidinyl]-N-hexyl-2,5-bis(methyloxy)benzenesulfonamide

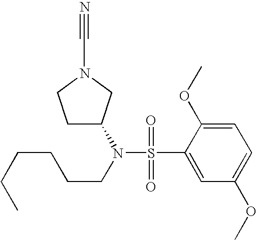

To a chilled (0° C.) solution of 1,1-dimethylethyl (3R)-3-({[2,5-bis(methyloxy)phenyl]sulfonyl}amino)-1-pyrrolidinecarboxylate (0.1766 g, 0.457 mmol) in DMF (3 ml) was added NaH (0.115 g, 4.57 mmol, 95%). The mixture was stirred for 5 minutes, then 1-bromohexane (0.28 ml, 1.995 mmol) was added. The mixture was warmed to room temperature then heated to 50° C. overnight. The mixture was cooled to room temperature, concentrated, and purified by preparatory HPLC (without TFA) to afford 1,1-dimethylethyl (3R)-3-[{[2,5-bis(methyloxy)phenyl]sulfonyl}(hexyl)amino]-1-pyrrolidinecarboxylate (0.0769 g, 0.163 mmol). LC-MS: m/z, 471 (M+H), rt 1.31 min. To this compound was added DCM (2 ml) and 4N HCl in 1,4-dioxane (1.0 ml, 4.00 mmol). The mixture was stirred at room temperature for 3 hours, then the solvent was evaporated. The residue was diluted with DCM (3 ml) and DIEA (0.09 mL, 0.515 mmol) and stirred for 10 min before BrCN (0.09 mL, 0.27 mmol) was added. The mixture was stirred at room temperature for 1 hour. PS-trisamine resin (1 equiv) was added to the mixture and stirred for 2 hours, then the mixture was filtered, concentrated and purified by preparatory HPLC (without TFA) to afford the title compound (0.0375 g). LC-MS: m/z, 396 (M+H), rt 1.12 min.

### Example 22

### N-[(3R)-1-Cyano-3-pyrrolidinyl]-N-(cyclohexylmethyl)-2,5-bis(methyloxy)benzenesulfonamide

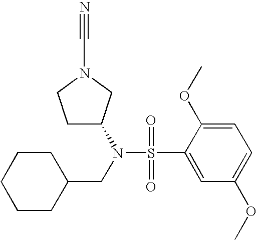

To a chilled (0° C.) solution of 1,1-dimethylethyl (3R)-3-({[2,5-bis(methyloxy)phenyl]sulfonyl}amino)-1-pyrrolidinecarboxylate (0.1788 g, 0.463 mmol) in DMF (3 ml) was added NaH (0.115 g, 4.57 mmol, 95%). The mixture was stirred for 5 minutes, then (bromomethyl)cyclohexane (0.28 ml, 2.007 mmol) was added. The mixture was warmed to room temperature then heated to 50° C. overnight. Reaction was then heated to 60° C. overnight. Cool to room temperature. More NaH (0.120 g, 4.75 mmol, 95%) and (bromomethyl)cyclohexane (0.20 ml, 1.43 mmol) were added and the mixture was reheated to 60° C. overnight. The mixture was cooled to room temperature, concentrated, and purified by preparatory HPLC (without TFA) to afford 1,1-dimethylethyl (3R)-3-[{[2,5-bis(methyloxy)phenyl]sulfonyl}(cyclohexylmethyl)amino]-1-pyrrolidinecarboxylate (0.037 g, 0.077 mmol). LC-MS: m/z, 483 (M+H), rt 1.31 min. To this compound was added DCM (2 ml) and 4N HCl in 1,4-dioxane (1.0 ml, 4.00 mmol). The mixture was stirred at room temperature for 2 hours, then the solvent was evaporated. The residue was diluted with DCM (3 ml) and DIEA (0.04 mL, 0.230 mmol) and stirred for 10 min before BrCN (0.04 mL, 0.120 mmol) was added. The mixture was stirred at room temperature for 3 hours. PS-trisamine resin (1 equiv) was added to the mixture and stirred for 2 hours, then the mixture was filtered, concentrated and purified by preparatory HPLC (without TFA) to afford the title compound (0.0204 g). LC-MS: m/z, 408 (M+H), rt 1.11 min.

### Example 23

### 2,5-Dibromo-N-[(4-bromophenyl)methyl]-N-[(3R)-1-cyano-3-pyrrolidinyl]benzenesulfonamide

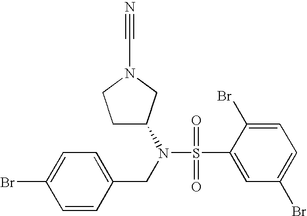

To a vial containing 1-bromo-4-(bromomethyl)benzene (0.1209 g, 0.480 mmol) and K₂CO₃ (0.25 g, 1.809 mmol) was added a solution of 1,1-dimethylethyl (3R)-3-{[(2,5-dibromophenyl)sulfonyl]amino}-1-pyrrolidinecarboxylate (0.048 g, 0.10 mmol) in acetone (2 ml). DMF (0.75 ml) was added to each vial. The mixture was shaken at room temperature for 2 days. PS-trisamine (0.293 g, 0.1 mmol, 3.41 mmol/g) was added to the mixture overnight. The mixture was then filtered and concentrated. The residue was taken up in DMSO and purified by preparatory HPLC (without TFA) to afford 1,1-dimethylethyl (3R)-3-{[(4-bromophenyl)methyl][(2,5-dibromophenyl)sulfonyl]amino}-1-pyrrolidinecarboxylate. To this compound was added DCM (1 ml) and 4N HCl in 1,4-dioxane (1.0 ml, 4.00 mmol). The mixture was shaken at room temperature for 2 hours, then the solvent was evaporated. The mixture was diluted with DCM (1.0 ml) mixed with DIEA (0.06 mL, 0.344 mmol), and was shaken for 20 min before BrCN (0.06 mL, 0.180 mmol) was added. The resultant mixture was stirred at room temperature for 6 hours. PS-trisamine resin (0.147 g, 0.500 mmol, 3.41 mmol/g) was added to the mixture for over the weekend, then the mixture was filtered, concentrated and purified by preparatory HPLC (without TFA) to afford the title compound (0.0147 g). LC-MS: m/z, 578 (M+H), rt 2.439 min.

Using the procedure described in Example 23, the following examples in Table 1 were synthesized, replacing 1-bromo-4-(bromomethyl)benzene with the relevant bromide.

TABLE 1

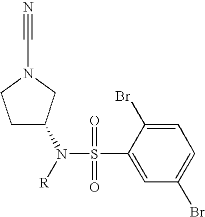

| | | | LC- |
|---|---|---|---|
| | | | MS: |
| | | Retention | m/z, |
| | | Time | # |
| Example | R | (min) | (M + H) |
| 24 | 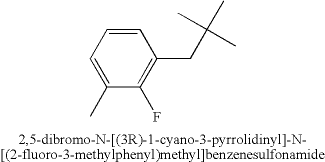 | 2.369 | 531 |
| 25 | 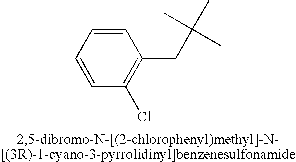 | 2.361 | 532.9 |
| 26 | 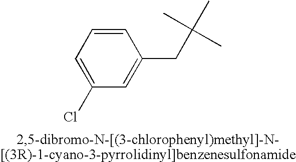 | 2.39 | 532.9 |
| 27 | 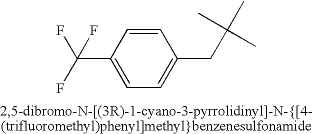 | 2.428 | 567 |
| 28 | 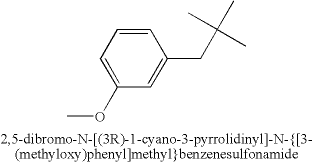 | 2.249 | 528.9 |
| 29 | 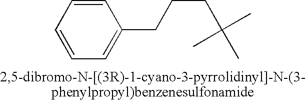 | 2.427 | 527 |
| 30 | 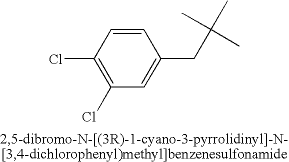 | 2.483 | 567.9 |
| 31 | 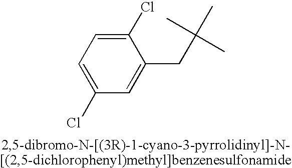 | 2.446 | 567.9 |
| 32 | 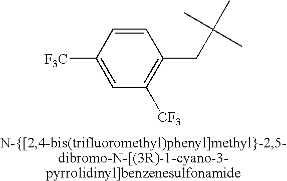 | 2.569 | 635 |
| 33 | 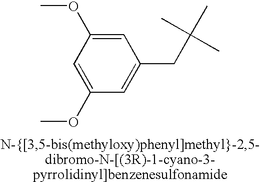 | 2.229 | 559 |
| 34 | 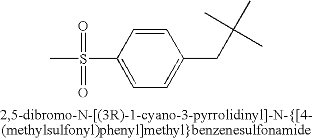 | 1.942 | 577 |
| 35 | 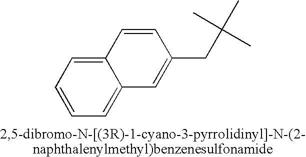 | 2.478 | 549 |
| 36 | 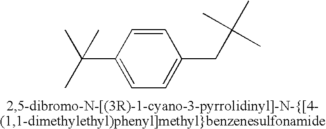 | 2.651 | 555 |
| 37 | 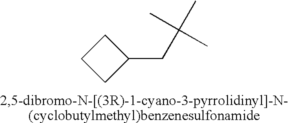 | 2.284 | 476.9 |
| 38 | 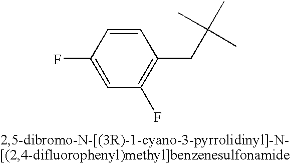 | 2.282 | 534.9 |
| 39 | 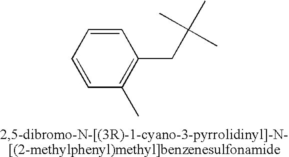 | 2.333 | 513 |
| 40 | 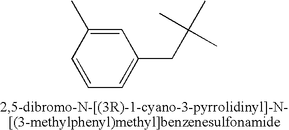 | 2.375 | 513 |
| 41 | 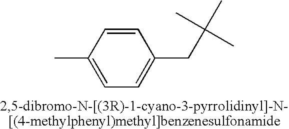 | 2.397 | 513 |

### Example 42

### N-[(4-Bromophenyl)methyl]-5-chloro-N-[(3R)-1-cyano-3-pyrrolidinyl]-2-(methyloxy)benzenesulfonamide

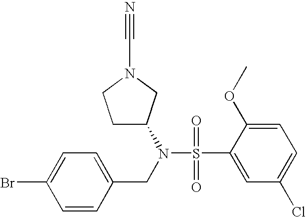

In a 1 dram vial was added 1,1-dimethylethyl (3R)-3-({[5-chloro-2-(methyloxy)phenyl]sulfonyl}amino)-1-pyrrolidinecarboxylate (0.065 g, 0.166 mmol) and K₂CO₃ (0.083 g, 0.600 mmol) in acetonitrile (2 ml). 1-Bromo-4-(bromomethyl)benzene (0.1265 g, 0.500 mmol) was added. The mixture was stirred at 60° C. overnight. MP-triamine (0.6 mmol) was added to the mixture overnight. The mixture was then filtered and concentrated. To this residue was added 4N HCl in 1,4-dioxane (1.0 ml, 4.00 mmol). The mixture was shaken at room temperature overnight, then the solvent was evaporated. The mixture was dissolved in DCM (2.0 ml) and DIEA (0.8 mmol) was added. The mixture was shaken for 3 hours before BrCN (0.4 mmol) was added. The resultant mixture was shaken at room temperature overnight. MP-triamine (0.111 g, 0.4 mmol) was added to the mixture for 3 hours, then the mixture was filtered, concentrated and purified by preparatory HPLC (without TFA) to afford the title compound (0.003 g). LC-MS: m/z, 483 (M+H), rt 1.45 min.

Using the procedure described in Example 42, the following examples in Table 2 were synthesized, replacing 1-bromo-4-(bromomethyl)benzene with the relevant bromide.

TABLE 2

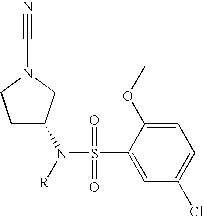

| | | | LC- |
|---|---|---|---|
| | | | MS: |
| | | Retention | m/z, |
| | | Time | # |
| Example | R | (min) | (M + H) |
| 43 | 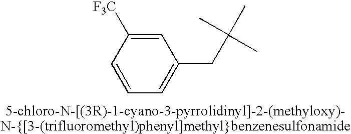 | 2.665 | 473.00 |
| 44 | 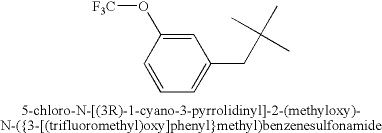 | 2.73 | 489.00 |
| 45 | 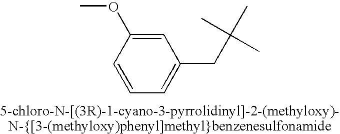 | 2.453 | 435.00 |
| 46 | 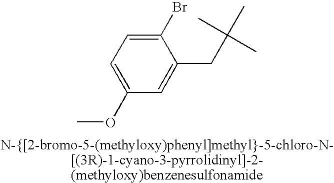 | 1.14 | 513.00 |
| 47 | 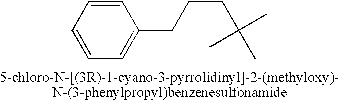 | 2.652 | 433.00 |
| 48 | 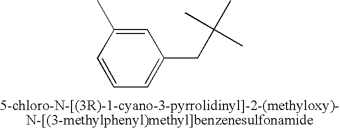 | 2.588 | 419.00 |
| 49 | 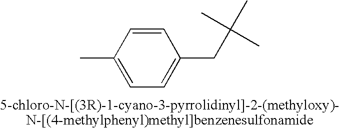 | 2.599 | 419.00 |
| 50 | 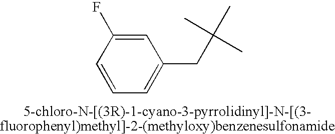 | 2.502 | 423.00 |
| 51 | 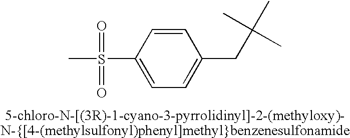 | 2.173 | 493.00 |
| 52 | 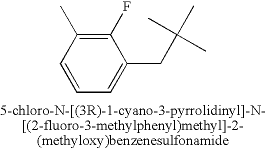 | 2.594 | 437.00 |
| 53 | 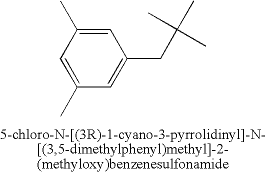 | 2.697 | 433.00 |
| 54 | 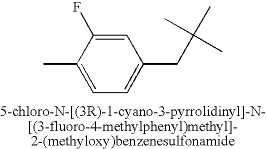 | 2.627 | 437.00 |
| 55 | 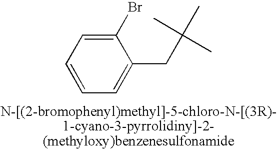 | 2.588 | 483.00 |
| 56 | 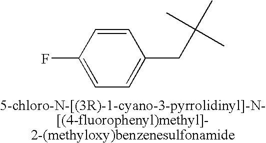 | 2.495 | 423.00 |
| 57 | 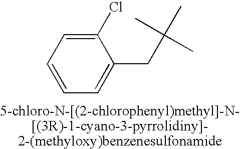 | 2.562 | 439.00 |
| 58 | 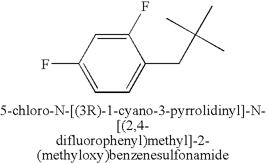 | 2.52 | 441.00 |
| 59 | 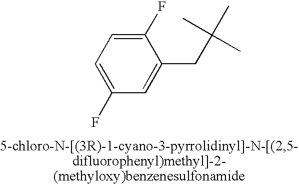 | 2.517 | 441.00 |
| 60 | 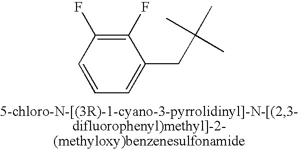 | 2.506 | 441.00 |

### Example 61

### N-[(4-Bromophenyl)methyl]-N-[(3R)-1-cyano-3-pyrrolidinyl]-2,5-bis(methyloxy)benzenesulfonamide

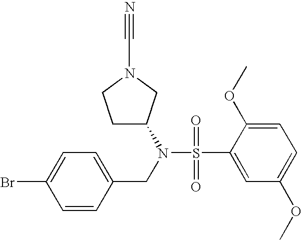

In a 1 dram vial was added 1,1-dimethylethyl (3R)-3-({[2,5-bis(methyloxy)phenyl]sulfonyl}amino)-1-pyrrolidinecarboxylate (0.065 g, 0.168 mmol) and K₂CO₃ (0.083 g, 0.600 mmol) in acetonitrile (2 ml). 1-bromo-4-(bromomethyl)benzene (0.1265 g, 0.500 mmol) was added. The mixture was stirred at 60° C. overnight. MP-triamine (0.6 mmol) was added to the mixture overnight. The mixture was then filtered, concentrated and purified by preparatory HPLC (without TFA) to afford 1,1-dimethylethyl (3R)-3-{{[2,5-bis(methyloxy)phenyl]sulfonyl}[(4-bromophenyl)methyl]amino}-1-pyrrolidinecarboxylate. To this compound was added DCM (2 ml) and 4N HCl in 1,4-dioxane (1.0 ml, 4.00 mmol). The mixture was shaken at room temperature overnight, then the solvent was evaporated. The residue was dissolved in DCM (2.8 ml) and DIEA (0.4 ml) was added. The mixture was shaken for 2 hours before BrCN (0.2 ml, 0.6 mmol) was added. The resultant mixture was shaken at room temperature overnight. PS-trisamine (0.260 g, 0.887 mmol) was added to the mixture for over the weekend, then the mixture was filtered, concentrated and purified by preparatory HPLC (without TFA) to afford the title compound (0.0473 g). LC-MS: m/z, 481 (M+H), rt 1.12 min.

Using the procedure described in Example 61, the following examples in Table 3 were synthesized, replacing 1-bromo-4-(bromomethyl)benzene with the relevant bromide.

TABLE 3

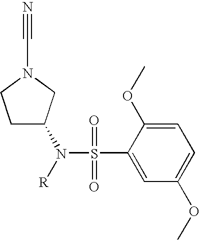

| | | | LC- |
|---|---|---|---|
| | | | MS: |
| | | Retention | m/z, |
| | | Time | # |
| Example | R | (min) | (M + H) |
| 62 | 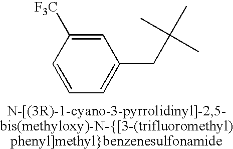 | 1.13 | 470 |
| 63 | 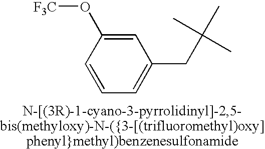 | 1.14 | 486 |
| 64 | 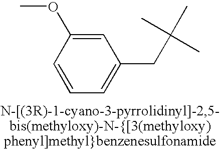 | 1.02 | 432 |
| 65 | 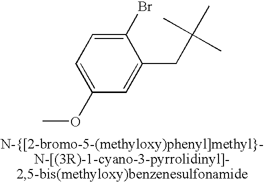 | 1.1 | 511 |
| 66 | 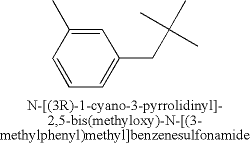 | 1.07 | 416 |
| 67 | 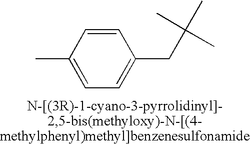 | 1.08 | 416 |
| 68 | 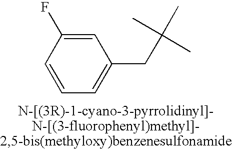 | 1.03 | 420 |
| 69 | 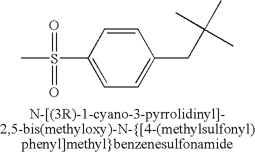 | 0.88 | 481 |
| 70 | 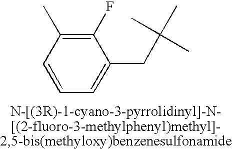 | 1.08 | 434 |
| 71 | 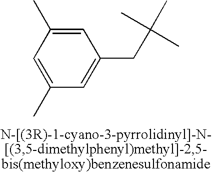 | 1.12 | 430 |
| 72 | 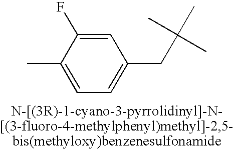 | 1.08 | 434 |
| 73 | 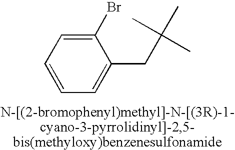 | 1.09 | 481 |
| 74 | 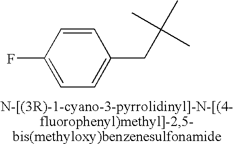 | 1.03 | 420 |
| 75 | 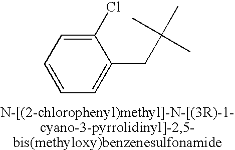 | 1.07 | 437 |
| 76 | 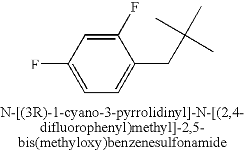 | 1.04 | 438 |
| 77 | 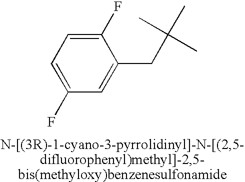 | 1.05 | 438 |
| 78 | 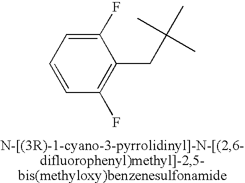 | 1 | 438 |
| 79 | 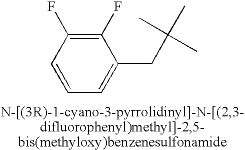 | 1.04 | 438 |

### Example 80

### 5-Bromo-N-[(4-bromophenyl)methyl]-N-[(3R)-1-cyano-3-pyrrolidinyl]-2-(methyloxy)benzenesulfonamide

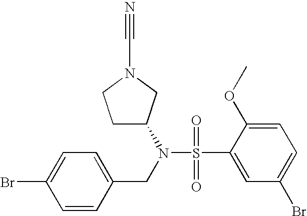

In a 1 dram vial was added 1,1-dimethylethyl (3R)-3-({[5-bromo-2-(methyloxy)phenyl]sulfonyl}amino)-1-pyrrolidinecarboxylate (0.065 g, 0.149 mmol) and K₂CO₃ (0.083 g, 0.600 mmol) in acetonitrile (2 ml). 1-bromo-4-(bromomethyl)benzene (0.1265 g, 0.500 mmol) was added. The mixture was stirred at 60° C. overnight. MP-triamine (0.6 mmol) was added to the mixture overnight. The mixture was then filtered, concentrated and purified by preparatory HPLC (without TFA) to afford 1,1-dimethylethyl (3R)-3-{{[5-bromo-2-(methyloxy)phenyl]sulfonyl}[(4-bromophenyl)methyl]amino}-1-pyrrolidinecarboxylate. To this compound was added DCM (2 ml) and 4N HCl in 1,4-dioxane (1.0 ml, 4.00 mmol). The mixture was shaken at room temperature overnight, then the solvent was evaporated. The residue was dissolved in DCM (2.8 ml) and DIEA (0.4 ml) was added. The mixture was shaken for 2 hours before BrCN (0.2 ml, 0.6 mmol) was added. The resultant mixture was shaken at room temperature overnight. PS-trisamine (0.260 g, 0.887 mmol) was added to the mixture for over the weekend, then the mixture was filtered, concentrated and purified by preparatory HPLC (without TFA) to afford the title compound (0.0241 g). LC-MS: m/z, 530 (M+H), rt 1.24 min.

Using the procedure described in Example 80, the following examples in Table 4 were synthesized, replacing 1-bromo-4-(bromomethyl)benzene with the relevant bromide.

TABLE 4

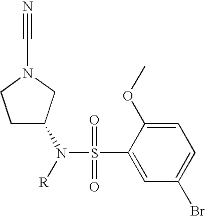

| | | | LC- |
|---|---|---|---|
| | | | MS: |
| | | Retention | m/z, |
| | | Time | # |
| Example | R | (min) | (M + H) |
| 81 | 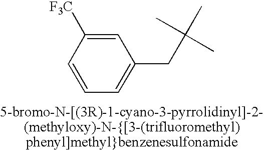 | 1.22 | 519 |
| 82 | 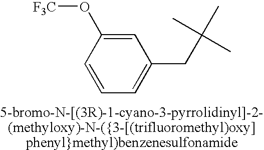 | 1.23 | 535 |
| 83 | 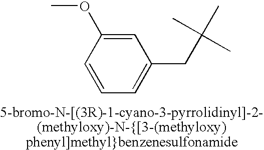 | 1.1 | 481 |
| 84 | 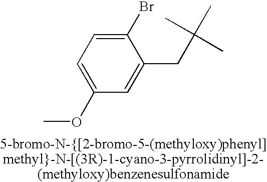 | 1.13 | 559.8 |
| 85 | 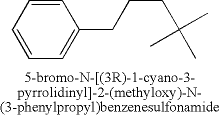 | 1.2 | 479 |
| 86 | 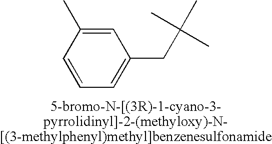 | 1.17 | 465 |
| 87 | 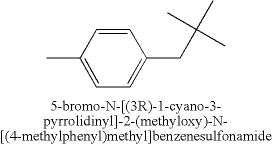 | 1.16 | 465 |
| 88 | 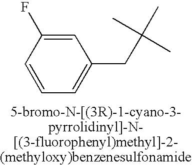 | 1.13 | 469 |
| 89 | 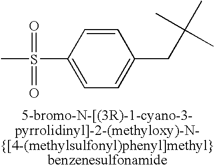 | 0.96 | 529 |
| 90 | 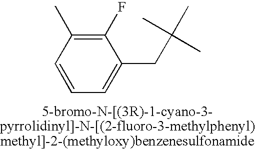 | 1.18 | 483 |
| 91 | 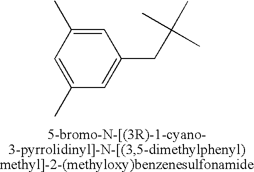 | 1.22 | 479.9 |
| 92 | 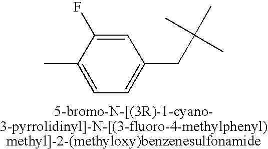 | 1.18 | 483 |
| 93 | 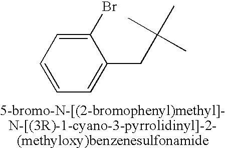 | 1.13 | 529.8 |
| 94 | 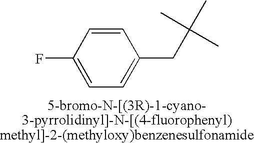 | 1.12 | 469 |
| 95 | 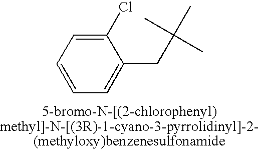 | 1.16 | 486 |
| 96 | 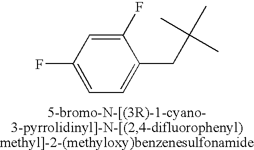 | 1.14 | 487 |
| 97 | 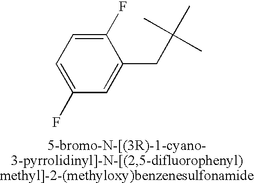 | 1.12 | 487 |
| 98 | 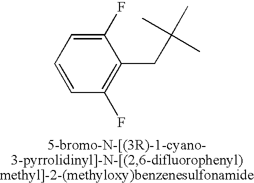 | 1.09 | 487 |
| 99 | 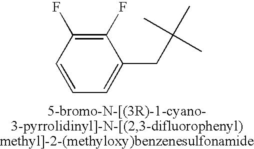 | 1.14 | 487 |

### Example 100

### N-[(3R)-1-Cyano-3-pyrrolidinyl]-N-(phenylmethyl)-2,5-bis[(2,2,2-trifluoroethyl)oxy]benzenesulfonamide

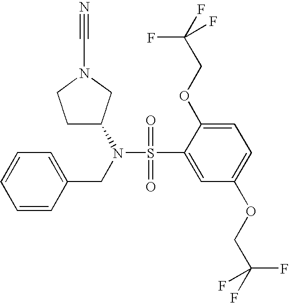

To solution of 1,1-dimethylethyl (3R)-3-[({2,5-bis[(2,2,2-trifluoroethyl)oxy]phenyl}sulfonyl)amino]-1-pyrrolidinecarboxylate (0.2009 g, 0.385 mmol) in acetonitrile (10 ml) was added K₂CO₃ (0.1603 g, 1.160 mmol) and (bromomethyl)benzene (0.14 ml, 1.177 mmol). The mixture was heated at 60° C. for overnight, after which the mixture was cooled to room temperature and concentrated. The residue was diluted with EtOAc (100 ml) and washed with 0.1M HCl (2×25 ml), sat NaHCO₃ (25 ml), and sat NaCl (25 ml). The organic layer was dried with MgSO₄, filtered and concentrated. The residue was purified by automated flash chromatography (40 g silica cartridge) to afford the 1,1-dimethylethyl (3R)-3-[({2,5-bis[(2,2,2-trifluoroethyl)oxy]phenyl}sulfonyl)(phenylmethyl)amino]-1-pyrrolidinecarboxylate (0.1987 g, 0.324 mmol). LC-MS: m/z, 613 (M+H), rt 1.34 min. To this compound was added DCM (2 ml) and 4N HCl in 1,4-dioxane (1.0 ml, 4.00 mmol). The mixture was stirred at room temperature for 2 hours, then the solvent was evaporated. The mixture was diluted with DCM (3.0 ml) and DIEA (0.17 mL, 0.973 mmol), and was stirred for 10 min before BrCN (0.16 mL, 0.487 mmol) was added. The resultant mixture was stirred at room temperature for 1 hour. PS-trisamine resin (1 equiv) was added to the mixture and stirred for 2 hours. The mixture was filtered, concentrated and purified by preparatory HPLC (without TFA) to afford the title compound (0.1215 g). LC-MS: m/z, 538 (M+H), rt 1.21 min.

### Example 101

### Methyl 3-{[[(3R)-1-cyano-3-pyrrolidinyl](phenylmethyl)amino]sulfonyl}-4-(methyloxy)benzoate

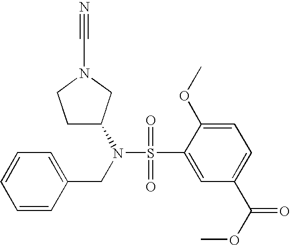

To solution of 1,1-dimethylethyl(3R)-3-[({2-(methyloxy)-5-[(methyloxy)carbonyl]phenyl}sulfonyl)amino]-1-pyrrolidinecarboxylate (0.2468 g, 0.595 mmol) in acetonitrile (10 ml) was added K₂CO₃ (0.257 g, 1.860 mmol) and (bromomethyl)benzene (0.22 ml, 1.852 mmol). The mixture was heated at 60° C. for overnight, after which the mixture was cooled to room temperature and concentrated. The residue was diluted with EtOAc (100 ml) and washed with 0.1M HCl (2×25 ml), sat NaHCO₃ (25 ml), and sat NaCl (25 ml). The organic layer was dried with MgSO₄, filtered and concentrated. The residue was purified by automated flash chromatography (40 g silica cartridge) to afford the 1,1-dimethylethyl (3R)-3-[({2-(methyloxy)-5-[(methyloxy)carbonyl]phenyl}sulfonyl)(phenylmethyl)amino]-1-pyrrolidinecarboxylate (0.1805 g, 0.358 mmol). LC-MS: m/z, 505 (M+H), rt 1.17 min. To this compound (0.090 g, 0.178 mmol) was added DCM (3 ml) and 4N HCl in 1,4-dioxane (0.75 ml). The mixture was stirred at room temperature for 3 hours, then the solvent was evaporated. The mixture was diluted with DCM (3.0 ml) and DIEA (0.10 mL, 0.573 mmol), and was stirred for 10 min before BrCN (0.1 mL, 0.300 mmol) was added. The resultant mixture was stirred at room temperature for 1 hour. PS-trisamine resin (1 equiv) was added to the mixture and stirred for 2 hours. The mixture was filtered, concentrated and purified by preparatory HPLC (without TFA) to afford the title compound (0.049 g). LC-MS: m/z, 430 (M+H), rt 1.02 min.

### Biological Background:

### Biological Assay(s)

The compounds according to formula I are cathepsin C inhibitors, which indirectly inhibit the activity of serine proteases that are activated by cathepsin C, such as NE. The compounds according to formula (IA) or (IB), therefore, are useful in the treatment of COPD and other conditions involving cathepsin C and/or such serine proteases. The biological activity of the compounds according to formula (IA) or (IB) can be determined using any suitable assay for determining the activity of a candidate compound as a cathepsin C inhibitor or for determining the ability of a candidate compound to prevent the cathepsin C mediated activation of certain serine proteases, as well as suitable tissue and in vivo models.

All examples were found to be cathepsin C inhibitors.

A. Transpeptidation of Leucine-Leucine-O-Methyl (LLOM) cell-based Luminescence Viability Assay

### Principle:

Cathepsin C has been shown to catalyze the transpeptidation of dipeptidyl methyl-O-esters within the lysosome of cell form the monocytic lineage like HL60, U937 or THP 1 causing a membranolythic effects that results in cell death (DL. Thiele P. Lipsky PNAS 1990 Vol. 87, pp. 83-87). This phenomenon was used to assess cellular Cathepsin C activity in the presence of our compounds.

Reagents:
Leucine-Leucine-OMethyl (Bachem, G-2550)
Iscove's Modified Dulbecco's Medium (IMDM) with L-Glutamine with 25 mMolar HEPES buffer (GSK Media Prep Lab)
Heat Inactivated Fetal Bovine Serum (GSK media prep lab)
HL60 (ATCC, CCL-240)
Dimethyl Sulfoxide (DMSO) (Sigma, D8418)
1M HEPES buffer solution (Gibco, 15630-080)
CellTiter-Glo Luminescent Cell Viability Assay (Promega, G7572, G7573)

### Protocol:

Pre-warm IMDM with 20% FBS media.
Count HL60 cells with Hemocytometer.
Spin cells down at 1200K for 5 min.
Re-suspend cells at 200K/ml in fresh pre-warmed media.

Dispense 100 ul/well of re-suspended cells in sterile black clear bottom polystyrene 96-well plate (Costar #3603).
Equilibrate cells for 30 min at 37° C., 5% CO2 incubator.
Add 1 ul of compound diluted at 100× concentrated DMSO. Compounds are ⅓ serial diluted in DMSO from 1 mM to 0.00 uM. Last row is DMSO only. Compounds are assayed in triplicate with final concentration 10 uM to 0.00 uM in wells.
Incubate plates at 37° C., 5% CO2 incubator for 30 min. Each plate is placed flat on shelf.
Add to each well 1 ul of 25 mM LLOM with 25 mM HEPES added in IMDM 20% FBS media to have 250 uM final in wells. LLOM solution is prepared fresh prior to addition.
Include a standard curve of compound GSK1473094A for QC.
Include a standard curve of LLOM for QC. LLOM is ½ serial diluted in IMDM with 20% FBS and 0.25 mM of HEPES media from 2 mM to 31.35 uM, Last row is only media.
Include three rows of cells without LLOM as 100% signal reference.
Incubate cells for 4 hours at 37° C., 5% CO2 incubator. Each plate is placed flat on shelf.
Thaw CellTiter-Glo buffer and substrate and equilibrate to room temperature.
Take the plates out of the incubator and lay flat on bench for 30 min to equilibrate to room temperature.
Add 100 ul of CellTiter-Glo to each well.
Rock plates for 5 min.
Read Luminescence (Wallace Envision reader and its software).
Plot and analyze data (using Graphpad Prism 4).

### B. Human Neutrophil Cathepsin C Assay

### Neutrophil Isolation:

Reagents:
Ficol-Paque Plus (Amersham Biosciences #17-1440-03)
PBS without calcium and magnesium, room temperature
Dextran T-500 (Pharmacia # 17-0320-01)—a 6% (w/v) solution in PBS without calcium and magnesium, stored in 25 ml aliqouts in freezer
Sterile water
Trypan blue
10×PBS without calcium and magnesium

Protocol:
At least 30 min. before blood is drawn, place 15 mL Ficol-Paque Plus into 50 mL Blue Max tubes.
Blood is collected, and each 25 mL of blood is layered over 15 mL Ficol-Paque Plus and centrifuged at 400 g at room temperature for 30 min. (brake is OFF).
Discard everything above the red cell fraction.
Red blood cell (RBC) pellets are resuspended to 35 mL in PBS w/o. Mix Dextran tubes by inversion, and add 12 mL to each blood tube. Mix RBC tubes by inversion and allow to stand undisturbed at room temperature for about 40 minutes (a clearly defined Richleau layer appears).
The layer above the RBC is collected, adjusted to 50 mL with PBS w/o, and centrifuged at 800 g at room temperature for 5 min. (brake can be on).
The sups are decanted and discarded down to about 3 mL, then the cells are gently dislodged and resuspended (in remaining supt.).
Lyse the RBC by adding 18 mL sterile water for 30 seconds at room temperature followed by 2 mL 10×PBS w/o.
The cell suspensions are adjusted to 50 mL with PBS w/o and centrifuged at 800 g for 5 min. at room temperature. Decant and discard sup.
Resuspend cells in PBS w/o (5 mL in each tube, pour two tubes together to make 10 mL). Remove 100 uL from tube of 10 mL, add it to 800 uL PBS w/o for counting, then bring the tubes up to 50 mL with PBS w/o. Centrifuge tubes once more at 800 g for 5 min.
To count cells, add 100 uL Trypan blue to the 900 uL tube. Place 10 uL on Hemacytometer. Count the cells in 5 different fields and average. This number×106 is your total number of cells.

### Assay:

### Reagents:

PBS: Dulbecco's Phosphorus Buffered Saline without calcium and magnesium
PBS/gelatin: Dulbecco's Phosphorus Buffered Saline without calcium and magnesium with 0.1% gelatin made from a stock of 3% gelatin (Sigma) which is boiled and frozen in aliquots.
96-welled v-bottom plates (polypropylene)
96-welled flat-bottom tissue culture plates
PMN isolated from human blood

### Protocol:

In a 96-welled v-bottom plate (polypropylene): 20 uL stock compound solution (10 mM in DMSO) added to wells in top row. DMSO added to alternating rows at 20 and 23 uL. Dilutions are made by placing 10 uL to the row below, mixing then repeating serially until reaching the bottom row using a multichannel pipettor.
Resuspend PMN in PBS/gelatin to a final concentration of 200,000 cells per mL. Plate in a 96-welled flat-bottom tissue culture plate 100 uL per well, giving a final concentration of 20,000 cells per well.
Add 1 uL of compound per well in triplicate, and mix for 5 min on a plate shaker. Incubate at 37C₅% CO2 for 30 min.
Add 5 uL of freshly diluted (H-Gly-Arg)₂ R110 Substrate (0.5 mM in PBS) and mix on a plate shaker for 5 min. Incubate at 37C, 5% CO2 for 3 hrs.
Read plate using the Analyst HD reader and Criterion Host software Excitation=485 nm, Emmission=530 nm, dichrioc mirror=505 nm.
Graph data using Graph Pad Prism non-linear regression curve fit analysis.

### C. Recombinant Cathepsin C In Vitro Assay:

The activity of recombinant human cathepsin C is measured by the cleavage of a fluorogenic substrate, H-Ser-Tyr-AMC. Briefly, 20 pM cathepsin C is incubated with test compound (e.g. inhibitor) in a buffer consisting of 50 mM sodium acetate, 30 mM sodium chloride, 1 mM CHAPS, 1 mM dithiothreitol, 1 mM EDTA, pH 5.5 at room temperature for one hour. After one hour of incubating test compound with cathepsin C, the activity assay is initiated by the addition of an equal volume of 0.010 mM H-Ser-Tyr-AMC in the same buffer. After one hour, the activity assay is stopped by the addition of 1/10 volume of 10 mM 2-Aldrithiol. The reaction product is measured on a fluorescence reader set at an excitation wavelength of 360 nm and emission wavelength of 460 nm and equipped with a 400 nm dichroic mirror.

These compound are believed to be useful in therapy as defined above and to not have unacceptable or untoward effects when used in compliance with a permited therapeutic regime.

The foregoing examples and assay have been set forth to illustrate the invention, not limit it. What is reserved to the inventors is to be determined by reference to the claims.

What is claimed is:

1. A compound of formula (I) 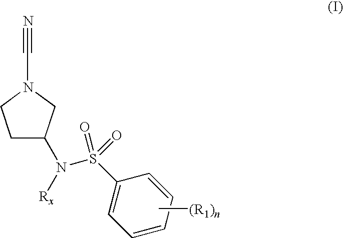
or a salt thereof wherein
Rₓ is C₁-C₆alkyl; or aryl or arylC₁-C₆alkyl where aryl is unsubstituted or substituted by halo, C₁-C₆alkyl, halo-substituted-C₁-C₆alkyl, C₁-C₆alkoxy optionally substituted with halo, or S(O)_{y} where y is 0-2;
n is 1-5,
each R₁ is independently halo; OR₂; C₁-C₁₀alkyl unsubstituted or substituted by halo; CN; C(O)NR₃R₄; NO₂; NHC(O)NR₅R₆; NR₇R₈; a heteraromatic ring containing 1-3 heteroatoms selected from the group consisting of N, O or S; aryl, unsubstituted or substituted by halo or C₁-C₆ alkyl; C₃-C₆cycloalkyl; C₄-C₆cycloalkenyl; C₁-C₁₀alkenyl; C₁-C₁₀alkynyl; or C(O)OR₂;
R₂ is H, C₁-C₆ alkyl, haloC₁-C₆ alkyl, C(O)R₉;
R₃ is H; C₁-C₆ alkyl; C₃-C₆cycloalkyl; or C₃-C₆heterocycloalkyl containing N, O or S and where when the ring heteroatom is N it is optionally substituted by CN;
R₄ is H, or C₁-C₆ alkyl;
R₅ and R₆ are independently H; C₁-C₆alkyl; aryl unsubstituted or substituted by halo, C₁-C₄alkoxy, C₁-C₄alkyl, or OR₂; C₁-C₁₀ alkyl unsubstituted or substituted by halo or C₁-C₄alkoxy; C₃-C₆cycloalkyl; C₃-C₆cycloalkylalkyl; C₁-C₁₀alkenyl; C₁-C₁₀alkynyl; C₃-C₆heteroycloalkyl; C₃-C₆heteroycloalkyl C₁-C₆alkyl; heteroarylC₁-C₆alkyl or heteroaryl wherein the heteroaryl ring is unsubstituted or substituted by halo, C₁-C₆ alkyl or halo-substituted-C₁-C₆ alkyl; or arylC₁-C₆alkyl wherein the aryl group is unsubstituted or substituted by halo, OR₂, or C(O);
R₇ and R₈ are independently H; C₁-C₁₀alkyl; arylC₁-C₆alkyl wherein the aryl group is unsubstituted or substituted by R₉, halo, or C₁-C₆alkyl; C₁-C₁₀alkyl substituted by C₃-C₆cycloalkyl, one or more OH groups, halo; heteroarylC₁-C₆alkyl; or heteroaryl; or
R₉ is OH, C₁-C₆ alkyoxy or NR_{2′}R_{3′}, wherein R_{2′} or R₃ are independently H or C₁-C₆alkyl.

2. A compound of formula I or a salt thereof according to claim 1 which is:
3-bromo-N-[(3R)-1-cyano-3-pyrrolidinyl]-N-methylbenzenesulfonamide
3-bromo-N-[(3R)-1-cyano-3-pyrrolidinyl]-N-(phenylmethyl)benzenesulfonamide
N-[(3R)-1-cyano-3-pyrrolidinyl]-N-methyl-2,5-bis(methyloxy)benzenesulfonamide
N-[(3R)-1-cyano-3-pyrrolidinyl]-2,5-bis(methyloxy)-N-(phenylmethyl)benzenesulfonamide
5-bromo-N-[(3R)-1-cyano-3-pyrrolidinyl]-N-methyl-2-(methyloxy)benzenesulfonamide
5-bromo-N-[(3R)-1-cyano-3-pyrrolidinyl]-2-(methyloxy)-N-(phenylmethyl)benzenesulfonamide
2,5-dichloro-N-[(3R)-1-cyano-3-pyrrolidinyl]-N-methylbenzenesulfonamide
2,5-dichloro-N-[(3R)-1-cyano-3-pyrrolidinyl]-N-(phenylmethyl)benzenesulfonamide
2-Chloro-N-[(3R)-1-cyano-3-pyrrolidinyl]-N-methyl-5-(trifluoromethyl)benzenesulfonamide
2-chloro-N-[(3R)-1-cyano-3-pyrrolidinyl]-N-(phenylmethyl)-5-(trifluoromethyl)benzenesulfonamide
2,5-dibromo-N-[(3R)-1-cyano-3-pyrrolidinyl]-N-methylbenzenesulfonamide
2,5-dibromo-N-[(3R)-1-cyano-3-pyrrolidinyl]-N-(phenylmethyl)benzenesulfonamide
2,5-dichloro-N-[(3R)-1-cyano-3-pyrrolidinyl]-N-propylbenzenesulfonamide
N-[(3R)-1-cyano-3-pyrrolidinyl]-2,5-bis(methyloxy)-N-propylbenzenesulfonamide
2,5-dibromo-N-[(3R)-1-cyano-3-pyrrolidinyl]-N-propylbenzenesulfonamide
2,5-dibromo-N-[(3R)-1-cyano-3-pyrrolidinyl]-N-(2-methylpropyl)benzenesulfonamide
2,5-dibromo-N-[(3R)-1-cyano-3-pyrrolidinyl]-N-(cyclohexylmethyl)benzenesulfonamide
2,5-dibromo-N-[(3R)-1-cyano-3-pyrrolidinyl]-N-hexylbenzenesulfonamide
N-[(3R)-1-cyano-3-pyrrolidinyl]-2,5-bis(methyloxy)-N-(2-methylpropyl)benzenesulfonamide
N-[(3R)-1-cyano-3-pyrrolidinyl]-N-ethyl-2,5-bis(methyloxy)benzenesulfonamide
N-[(3R)-1-cyano-3-pyrrolidinyl]-N-hexyl-2,5-bis(methyloxy)benzenesulfonamide
N-[(3R)-1-cyano-3-pyrrolidinyl]-N-(cyclohexylmethyl)-2,5-bis(methyloxy)benzenesulfonamide
2,5-dibromo-N-[(4-bromophenyl)methyl]-N-[(3R)-1-cyano-3-pyrrolidinyl]benzenesulfonamide
2,5-dibromo-N-[(3R)-1-cyano-3-pyrrolidinyl]-N-[(2-fluoro-3-methylphenyl)methyl]benzenesulfonamide
2,5-dibromo-N-[(2-chlorophenyl)methyl]-N-[(3R)-1-cyano-3-pyrrolidinyl]benzenesulfonamide
2,5-dibromo-N-[(3-chlorophenyl)methyl]-N-[(3R)-1-cyano-3-pyrrolidinyl]benzenesulfonamide
2,5-dibromo-N-[(3R)-1-cyano-3-pyrrolidinyl]-N-{[4-(trifluoromethyl)phenyl]methyl}benzenesulfonamide
2,5-dibromo-N-[(3R)-1-cyano-3-pyrrolidinyl]-N-{[3-(methyloxy)phenyl]methyl}benzenesulfonamide
2,5-dibromo-N-[(3R)-1-cyano-3-pyrrolidinyl]-N-(3-phenylpropyl)benzenesulfonamide
2,5-dibromo-N-[(3R)-1-cyano-3-pyrrolidinyl]-N-[(3,4-dichlorophenyl)methyl]benzenesulfonamide
2,5-dibromo-N-[(3R)-1-cyano-3-pyrrolidinyl]-N-[(2,5-dichlorophenyl)methyl]benzenesulfonamide
N-{[2,4-bis(trifluoromethyl)phenyl]methyl}-2,5-dibromo-N-[(3R)-1-cyano-3-pyrrolidinyl]benzenesulfonamide
N-{[3,5-bis(methyloxy)phenyl]methyl}-2,5-dibromo-N-[(3R)-1-cyano-3-pyrrolidinyl]benzenesulfonamide
2,5-dibromo-N-[(3R)-1-cyano-3-pyrrolidinyl]-N-{[4-(methylsulfonyl)phenyl]methyl}benzenesulfonamide
2,5-dibromo-N-[(3R)-1-cyano-3-pyrrolidinyl]-N-(2-naphthalenylmethyl)benzenesulfonamide
2,5-dibromo-N-[(3R)-1-cyano-3-pyrrolidinyl]-N-{[4-(1,1-dimethylethyl)phenyl]methyl}benzenesulfonamide
2,5-dibromo-N-[(3R)-1-cyano-3-pyrrolidinyl]-N-(cyclobutylmethyl)benzenesulfonamide
2,5-dibromo-N-[(3R)-1-cyano-3-pyrrolidinyl]-N-[(2,4-difluorophenyl)methyl]benzenesulfonamide
2,5-dibromo-N-[(3R)-1-cyano-3-pyrrolidinyl]-N-[(2-methylphenyl)methyl]benzenesulfonamide
2,5-dibromo-N-[(3R)-1-cyano-3-pyrrolidinyl]-N-[(3-methylphenyl)methyl]benzenesulfonamide
2,5-dibromo-N-[(3R)-1-cyano-3-pyrrolidinyl]-N-[(4-methylphenyl)methyl]benzenesulfonamide
N-[(4-bromophenyl)methyl]-5-chloro-N-[(3R)-1-cyano-3-pyrrolidinyl]-2-(methyloxy)benzenesulfonamide
5-chloro-N-[(3R)-1-cyano-3-pyrrolidinyl]-2-(methyloxy)-N-{[3-(trifluoromethyl)phenyl]methyl}benzenesulfonamide
5-chloro-N-[(3R)-1-cyano-3-pyrrolidinyl]-2-(methyloxy)-N-({3-[(trifluoromethyl)oxy]phenyl}methyl)benzenesulfonamide
5-chloro-N-[(3R)-1-cyano-3-pyrrolidinyl]-2-(methyloxy)-N-{[3-(methyloxy)phenyl]methyl}benzenesulfonamide
N-{[2-bromo-5-(methyloxy)phenyl]methyl}-5-chloro-N-[(3R)-1-cyano-3-pyrrolidinyl]-2-(methyloxy)benzenesulfonamide
5-chloro-N-[(3R)-1-cyano-3-pyrrolidinyl]-2-(methyloxy)-N-(3-phenylpropyl)benzenesulfonamide
5-chloro-N-[(3R)-1-cyano-3-pyrrolidinyl]-2-(methyloxy)-N-[(3-methylphenyl)methyl]benzenesulfonamide
5-chloro-N-[(3R)-1-cyano-3-pyrrolidinyl]-2-(methyloxy)-N-[(4-methylphenyl)methyl]benzenesulfonamide
5-chloro-N-[(3R)-1-cyano-3-pyrrolidinyl]-N-[(3-fluorophenyl)methyl]-2-(methyloxy)benzenesulfonamide
5-chloro-N-[(3R)-1-cyano-3-pyrrolidinyl]-2-(methyloxy)-N-{[4-(methylsulfonyl)phenyl]methyl}benzenesulfonamide
5-chloro-N-[(3R)-1-cyano-3-pyrrolidinyl]-N-[(2-fluoro-3-methylphenyl)methyl]-2-(methyloxy)benzenesulfonamide
5-chloro-N-[(3R)-1-cyano-3-pyrrolidinyl]-N-[(3,5-dimethylphenyl)methyl]-2-(methyloxy)benzenesulfonamide
5-chloro-N-[(3R)-1-cyano-3-pyrrolidinyl]-N-[(3-fluoro-4-methylphenyl)methyl]-2-(methyloxy)benzenesulfonamide
N-[(2-bromophenyl)methyl]-5-chloro-N-[(3R)-1-cyano-3-pyrrolidinyl]-2-(methyloxy)benzenesulfonamide
5-chloro-N-[(3R)-1-cyano-3-pyrrolidinyl]-N-[(4-fluorophenyl)methyl]-2-(methyloxy)benzenesulfonamide
5-chloro-N-[(2-chlorophenyl)methyl]-N-[(3R)-1-cyano-3-pyrrolidinyl]-2-(methyloxy)benzenesulfonamide
5-chloro-N-[(3R)-1-cyano-3-pyrrolidinyl]-N-[(2,4-difluorophenyl)methyl]-2-(methyloxy)benzenesulfonamide
5-chloro-N-[(3R)-1-cyano-3-pyrrolidinyl]-N-[(2,5-difluorophenyl)methyl]-2-(methyloxy)benzenesulfonamide
5-chloro-N-[(3R)-1-cyano-3-pyrrolidinyl]-N-[(2,3-difluorophenyl)methyl]-2-(methyloxy)benzenesulfonamide
N-[(4-bromophenyl)methyl]-N-[(3R)-1-cyano-3-pyrrolidinyl]-2,5-bis(methyloxy)benzenesulfonamide
N-[(3R)-1-cyano-3-pyrrolidinyl]-2,5-bis(methyloxy)-N-{[3-(trifluoromethyl)phenyl]methyl}benzenesulfonamide
N-[(3R)-1-cyano-3-pyrrolidinyl]-2,5-bis(methyloxy)-N-({3-[(trifluoromethyl)oxy]phenyl}methyl)benzenesulfonamide
N-[(3R)-1-cyano-3-pyrrolidinyl]-2,5-bis(methyloxy)-N-{[3-(methyloxy)phenyl]methyl}benzenesulfonamide
N-{[2-bromo-5-(methyloxy)phenyl]methyl}-N-[(3R)-1-cyano-3-pyrrolidinyl]-2,5-bis(methyloxy)benzenesulfonamide
N-[(3R)-1-cyano-3-pyrrolidinyl]-2,5-bis(methyloxy)-N-[(3-methylphenyl)methyl]benzenesulfonamide
N-[(3R)-1-cyano-3-pyrrolidinyl]-2,5-bis(methyloxy)-N-[(4-methylphenyl)methyl]benzenesulfonamide
N-[(3R)-1-cyano-3-pyrrolidinyl]-N-[(3-fluorophenyl)methyl]-2,5-bis(methyloxy)benzenesulfonamide
N-[(3R)-1-cyano-3-pyrrolidinyl]-2,5-bis(methyloxy)-N-{[4-(methylsulfonyl)phenyl]methyl}benzenesulfonamide
N-[(3R)-1-cyano-3-pyrrolidinyl]-N-[(2-fluoro-3-methylphenyl)methyl]-2,5-bis(methyloxy)benzenesulfonamide
N-[(3R)-1-cyano-3-pyrrolidinyl]-N-[(3,5-dimethylphenyl)methyl]-2,5-bis(methyloxy)benzenesulfonamide
N-[(3R)-1-cyano-3-pyrrolidinyl]-N-[(3-fluoro-4-methylphenyl)methyl]-2,5-bis(methyloxy)benzenesulfonamide
N-[(2-bromophenyl)methyl]-N-[(3R)-1-cyano-3-pyrrolidinyl]-2,5-bis(methyloxy)benzenesulfonamide
N-[(3R)-1-cyano-3-pyrrolidinyl]-N-[(4-fluorophenyl)methyl]-2,5-bis(methyloxy)benzenesulfonamide
N-[(2-chlorophenyl)methyl]-N-[(3R)-1-cyano-3-pyrrolidinyl]-2,5-bis(methyloxy)benzenesulfonamide
N-[(3R)-1-cyano-3-pyrrolidinyl]-N-[(2,4-difluorophenyl)methyl]-2,5-bis(methyloxy)benzenesulfonamide
N-[(3R)-1-cyano-3-pyrrolidinyl]-N-[(2,5-difluorophenyl)methyl]-2,5-bis(methyloxy)benzenesulfonamide
N-[(3R)-1-cyano-3-pyrrolidinyl]-N-[(2,6-difluorophenyl)methyl]-2,5-bis(methyloxy)benzenesulfonamide
N-[(3R)-1-cyano-3-pyrrolidinyl]-N-[(2,3-difluorophenyl)methyl]-2,5-bis(methyloxy)benzenesulfonamide
5-bromo-N-[(4-bromophenyl)methyl]-N-[(3R)-1-cyano-3-pyrrolidinyl]-2-(methyloxy)benzenesulfonamide
5-bromo-N-[(3R)-1-cyano-3-pyrrolidinyl]-2-(methyloxy)-N-{[3-(trifluoromethyl)phenyl]methyl}benzenesulfonamide
5-bromo-N-[(3R)-1-cyano-3-pyrrolidinyl]-2-(methyloxy)-N-({3-[(trifluoromethyl)oxy]phenyl}methyl)benzenesulfonamide
5-bromo-N-[(3R)-1-cyano-3-pyrrolidinyl]-2-(methyloxy)-N-{[3-(methyloxy)phenyl]methyl}benzenesulfonamide
5-bromo-N-{[2-bromo-5-(methyloxy)phenyl]methyl}-N-[(3R)-1-cyano-3-pyrrolidinyl]-2-(methyloxy)benzenesulfonamide
5-bromo-N-[(3R)-1-cyano-3-pyrrolidinyl]-2-(methyloxy)-N-(3-phenylpropyl)benzenesulfonamide
5-bromo-N-[(3R)-1-cyano-3-pyrrolidinyl]-2-(methyloxy)-N-[(3-methylphenyl)methyl]benzenesulfonamide
5-bromo-N-[(3R)-1-cyano-3-pyrrolidinyl]-2-(methyloxy)-N-[(4-methylphenyl)methyl]benzenesulfonamide
5-bromo-N-[(3R)-1-cyano-3-pyrrolidinyl]-N-[(3-fluorophenyl)methyl]-2-(methyloxy)benzenesulfonamide
5-bromo-N-[(3R)-1-cyano-3-pyrrolidinyl]-2-(methyloxy)-N-{[4-(methylsulfonyl)phenyl]methyl}benzenesulfonamide
5-bromo-N-[(3R)-1-cyano-3-pyrrolidinyl]-N-[(2-fluoro-3-methylphenyl)methyl]-2-(methyloxy)benzenesulfonamide
5-bromo-N-[(3R)-1-cyano-3-pyrrolidinyl]-N-[(3,5-dimethylphenyl)methyl]-2-(methyloxy)benzenesulfonamide
5-bromo-N-[(3R)-1-cyano-3-pyrrolidinyl]-N-[(3-fluoro-4-methylphenyl)methyl]-2-(methyloxy)benzenesulfonamide
5-bromo-N-[(2-bromophenyl)methyl]-N-[(3R)-1-cyano-3-pyrrolidinyl]-2-(methyloxy)benzenesulfonamide
5-bromo-N-[(3R)-1-cyano-3-pyrrolidinyl]-N-[(4-fluorophenyl)methyl]-2-(methyloxy)benzenesulfonamide
5-bromo-N-[(2-chlorophenyl)methyl]-N-[(3R)-1-cyano-3-pyrrolidinyl]-2-(methyloxy)benzenesulfonamide
5-bromo-N-[(3R)-1-cyano-3-pyrrolidinyl]-N-[(2,4-difluorophenyl)methyl]-2-(methyloxy)benzenesulfonamide
5-bromo-N-[(3R)-1-cyano-3-pyrrolidinyl]-N-[(2,5-difluorophenyl)methyl]-2-(methyloxy)benzenesulfonamide
5-bromo-N-[(3R)-1-cyano-3-pyrrolidinyl]-N-[(2,6-difluorophenyl)methyl]-2-(methyloxy)benzenesulfonamide
5-bromo-N-[(3R)-1-cyano-3-pyrrolidinyl]-N-[(2,3-difluorophenyl)methyl]-2-(methyloxy)benzenesulfonamide
N-[(3R)-1-cyano-3-pyrrolidinyl]-N-(phenylmethyl)-2,5-bis[(2,2,2-trifluoroethyl)oxy]benzenesulfonamide; or
methyl 3-{[[(3R)-1-cyano-3-pyrrolidinyl](phenylmethyl)amino]sulfonyl}-4-(methyloxy)benzoate.

3. A method of treating chronic obstructive pulmonary disease in a patient comprising administering an effective amount of a compound of formula I according to claim 1 or a pharmaceutically acceptable salt thereof, either neat or admixed with a pharmaceutically acceptable excipient.

4. A pharmaceutical composition comprising a compound of formula (I) or a pharmaceutically acceptable salt thereof according to claim 1, and one or more of pharmaceutically acceptable carriers, diluents and excipients.
